SEQUENCE LISTING (2 sequences)

SEQ ID NO: 1 - DNA, 750 bp, Streptomyces koganeinsis
  misc_feature: Streptomyces koganeinsis hyaluronidase
atgccggttgcgcgtcgtctgttcctgggctctttcaccgcgggcgcggttaccgttgcgaccgcggcggcgaccggcaccgcgtctgcggcgggcgaaaacggcgcgaccaccaccttcgacggcccggttgcggcggaacgtttctctgcggacaccaccctggaagcggcgttcctgaaaaccacctctgaaaccaaccacgcggcgaccatctaccaggcgggcacctctggcgacggcgcggcgctgaacgttatctctgacaacccgggcacctctgcgatgtacctgtctggcaccgaaaccgcgcgtggcaccctgaaaatcacccaccgtggctacgcggacggctctgacaaagacgcggcggcgctgtctctggacctgcgtgttgcgggcaccgcggcgcagggcatctacgttaccgcgaccaacggcccgaccaaaggcaacctgatcgcgctgcgtaacaacaccggcctggacgacttcgttgttaaaggcaccggccgtatcggcgttggcatcgaccgtgcggcgaccccgcgtgcgcaggttcacatcgttcagcgtggcgacgcgctggcggcgctgctggttgaaggctctgttcgtatcggcaacgcggcgaccgttccgacctctgttgactcttctggcggcggcgcgctgtacgcgtctggcggcgcgctgctgtggcgtggctctaacggcaccgttaccaccatcgcgccggcgtaatagtga

SEQ ID NO: 2 - DNA, 783 bp, Streptomyces omiyaensis
  misc_feature: Streptomyces omiyaensis trypsin-like protease
atgcagaaaaaccgtctggttcgtaccctgcagaaactggcggcggcgggcgcggttgcgctggcggcgctgtctctgcagccggtttcttctgcgaccgcggcgccgaacccggttgttggcggcacccgtgcggcgcagggcgaatttccgtggatggttcgtctgtctatgggctgcggcggctctctgatcaccccgcaggttgttctgaccgcggcgcactgcgttggcgcgaccggcaacaacacctctatcaccgcgaccgcgggcgttgttgacctgcagtcttcttctgcgatcaaagttcgttctaccaaaatctaccgtgcgccgggctacaacggcaaaggcaaagactgggcgctgatcaaactggcgtctccgatcacctctctgccgaccctgaaactggcggaaaccaccgcgtacaactctggcaccttcaccgttgcgggctggggcgcggcgcgtgaaggcggcggccagcagcgttacctgctgaaagcgaacgttccgttcgtttctgacgcgtcttgccaggcgtcttacggctctgacctggttccgtctgaagaaatctgcgcgggctacccgcagggcggcgttgacacctgccagggcgactctggcggcccgatgttccgtaaagacaacgcgggcgcgtgggttcaggttggcatcgtttcttggggccagggctgcgcgcgtccggactacccgggcgtttacaccgaagtttctaccttcgcggcggcgatcaaatctgcggcggcgaccctg

(19) United States
(12) Patent Application Publication
(10) Pub. No.: US 2022/0193149 A1
(43) Pub. Date: Jun. 23, 2022

(54) COMPOSITIONS AND METHODS FOR TARGETING TUMOR-ASSOCIATED EXTRACELLULAR MATRIX COMPONENTS TO IMPROVE DRUG DELIVERY

(71) Applicant: City of Hope, Duarte, CA (US)

(72) Inventors: Edwin Ramos Manuel, Fontana, CA (US); Don J. Diamond, Glendora, CA (US)

(21) Appl. No.: 17/611,393

(22) PCT Filed: May 15, 2020
(86) PCT No.: PCT/US2020/033227

§ 371 Date: Nov. 15, 2021

Related U.S. Application Data
(60) Provisional application No. 62/848,873, filed on May 16, 2019.

(51) Int. Cl. A61K 35/74; A61K 38/14; A61K 38/21; A61K 38/50; A61K 45/06; C12N 15/74; C12N 9/26; C12N 9/52

(52) U.S. Cl. CPC A61K 35/74; A61K 38/14; A61K 38/212; A61K 38/50; C12N 15/11; C12N 15/74; C12N 9/2474; C12N 9/52; A61K 45/06

(57) ABSTRACT

Provided herein are compositions and methods to treat tumors that include attenuated facultative anaerobic bacterium. The bacterium includes a nucleic acid molecule encoding a recombinant extracellular matrix degrading enzyme operably linked to a promoter.

### CROSS-REFERENCED APPLICATIONS

This application claims priority benefit to U.S. provisional 62/848,873 filed May 16, 2019, which is incorporated herein in its entirety.

### STATEMENT AS TO RIGHTS TO INVENTIONS MADE UNDER FEDERALLY SPONSORED RESEARCH AND DEVELOPMENT

This invention was made with government support under grant nos. P30 CA033572 awarded by the National Cancer Institute. The government has certain rights in the invention.

### SEQUENCE LISTING

The instant application contains a Sequence Listing, which has been submitted electronically in ASCII format and is hereby incorporated by reference in its entirety. Said ASCII copy, created on May 15, 2020 is named 048440-723001WO_SEQUENCE_LISTING_ST25.txt and is 2,799 bytes in size.

### BACKGROUND

Hyaluronan, also known as hHA, is a component of pancreatic ductal adenocarcinoma (PDAC) stroma that is expressed at extremely high levels in the extracellular matrix (ECM), resulting in a biophysical barrier that significantly increases interstitial fluidic pressure, compresses blood vessels and hinders effective drug delivery. While PDAC tumors have the greatest incidence of HA overexpression in patients (>95%), other cancer types such as breast and prostate cancer express high levels. Thus, agents to degrade tumor-derived HA, and other overexpressed ECM components, to improve drug delivery and efficacy has been an area of extensive research. Various forms of bovine hyaluronidase and human PH20 hyaluronidase have been utilized to enhance the delivery of chemotherapy into solid tumors. However, because these enzymes are delivered systemically and their activity is not restricted to only tumor tissue, significant adverse events have been observed relating to HA depletion in joints and other organs, requiring lower doses or co-administration with additional agents to minimize these stresses. (See 4-22).

### BRIEF SUMMARY

In view of the foregoing, there remains the need for new agents and treatment strategies to improve overall survival for patients with cancer. The present disclosure addresses this need, and provides additional benefits as well.

In an aspect, provided herein is an attenuated facultative anaerobic bacterium including a nucleic acid molecule encoding a recombinant extracellular matrix degrading enzyme operably linked to a promoter.

In an aspect, provided herein is a method of treating a tumor in a subject including administering to the subject an effective amount of an attenuated facultative anaerobic bacterium that includes a nucleic acid molecule encoding a recombinant extracellular matrix degrading enzyme operably linked to a promoter.

In an aspect, provided herein is a method of treating a tumor in a subject including administering to the subject an effective amount of an attenuated facultative anaerobic bacterium that includes a nucleic acid molecule encoding a recombinant extracellular matrix degrading enzyme operably linked to a promoter.

### BRIEF DESCRIPTION OF THE DRAWINGS

FIGS. 1A-1C presents data demonstrating transgene stability and expression of Streptomyces koganeiensis hyaluronidase (bHs) by attenuated ST strains. FIG. 1A presents polymerase chain reaction (PCR) experiments to detect for the bHs transgene contained within an inducible pBAD vector (pBAD-bHs) transformed into indicated ST strains. Representative colony PCRs shown from ≥8 colonies per transformed strain. A positive PCR control using ST-specific attB primers was performed for each colony. E. coli (BL21) transformed with pBAD-bHs is used as a positive PCR control for bHs and negative control for ST attB. FIG. 1B shows ST strains retaining the bHs transgene were cultured in Luria Broth (LB) containing 0% (uninduced) or 2% (induced) L-arabinose and cultured for 3 hours at 37° C. Lysates from ˜5×10⁷ colony forming units (CFUs) were run on a 4-20% polyacrylamide gradient gel and subjected to coomassie blue staining (CB) and western blot analysis against an amino terminal His-tag fused to bHs (α-His). Predicted bHs size ˜27 kDa (arrow). L=protein ladder. FIG. 1C shows that ST strains encoding His-tagged bHs were cultured in LB media containing 2% L-arabinose and immunostained (α-His) to determine localization of bHs. The cytosol is imaged by staining genomic DNA with DAPI. His-tagged bHs expression by SL7207 colocalizes with DAPI, indicating that bHs is localized in the cytosol. In χ8431, His-tagged bHs expression is punctate (arrows) and surrounding the nucleus, indicating bHs protein is contained within inclusion bodies. In χ8429 and χ8768, bHs is observed to be localized to the membrane (dotted line) outside of the cytoplasm. All data presented are representative of ≥3 experiments.

FIGS. 2A-2E demonstrate growth kinetics and viability of bHs-expressing ST strains. FIGS. 2A-C show optical density readings (OD₆₀₀) for uninduced (solid blue circles) and induced (open red circles) ST strains SL7207 (FIG. 2A), χ8429 (FIG. 2B) and χ8768 (FIG. 2C) transformed with the pBAD-bHs construct. Cultures were done in triplicate and error bars represent standard error of the mean. FIG. 2D are growth curves of induced bHs-expressing strains are compared. *p<0.05 by ANOVA. FIG. 2E is data showing bacterial cells from uninduced (−) and induced (+2% L-arabinose) cultures of SL7207-bHs and χ8768-bHs were stained at indicated time points (4 and 24 hours) with acridine orange to indicate live bacterium and imaged by fluorescence microscopy at 100× magnification. Under induced conditions (+) the majority of SL7207 at 4 and 24 hours post-induction are observed in aggregates and to have minimal (dim) staining (arrows), indicating dead bacterium. χ8768-bHs stain strongly with acridine orange, suggesting high viability following induction. All data are representative of ≥3 experiments.

FIGS. 3A-3D demonstrate functional analysis of bHs-expressing ST strains. FIG. 3A shows bHs-expressing strains were cultured in LB broth containing indicated percentages of L-arabinose for 3 hours at 37° C. 1×10⁸ CFUs were plated onto LB-hyaluronan-BSA agar plates overnight and then flushed with 2N acetic acid. Plates were imaged on a black background to visualize areas of clearing, indicating hyaluronan breakdown. FIG. 3B shows 1×10⁸ CFUs of ST-bHs strains were added to LB containing 0% or 2% L-arabinose and 0.4 mg/mL HA. The cetyltrimethylammonium bromide turbidimetric method (CTM) was used to determine rate of HA breakdown over 24 hours (OD₆₀₀) for pBAD-bHs-transformed SL7207, (FIG. 3C) χ8768 and (FIG. 3D) χ8729. Error bars=standard error of the mean. All data are representative of ≥3 experiments.

FIG. 4 demonstrates that induced χ8768-bHs effectively depletes tumor-derived hyaluronan. χ8768-bHs was grown for 3 hours in LB media containing 0% (uninduced) or 2% (induced) L-arabinose. 1×10⁸ CFUs were then co-incubated with serial sections of PANC-1 tumor tissue overnight. HA was detected using biotinylated HA-binding protein (HABP) followed by incubation with Vectastain strepavidin-HRP and ImmPACT DAB substrate. Serial sections incubated with PBS serve as an HA-positive control and specificity of HABP was confirmed through overnight incubation with 10 U/mL bovine hyaluronidase (Bov. Hs). Scale bar=75 uM. All images are representative of ≥3 experiments.

FIGS. 5A-5D demonstrate systemic delivery of χ8768-bHs effectively degrades HA within orthotopic PANC-1 tumors. Uninduced χ8768-bHs (2.5×10⁶ CFU) was injected intravenously (i.v.) into NSG mice bearing orthotopic PANC-1 tumors (>250 mm³). After 48 hours, mice were then administered (FIG. 5A) PBS (uninduced) or (FIG. 5B) 250 mg L-arabinose (induced) by intraperitoneal injection. Tumors were isolated 16 hours later, sectioned and stained for ST and HA for subsequent immunofluorescence imaging at 10× and 100× magnification (with nuclear staining using DAPI present in overlays). Under uninduced conditions in FIG. 5A, areas colonized by ST also show presence of HA, indicating no depletion of HA. Under induced conditions in FIG. 5B, ST colonization is associated with absence of HA staining, suggesting HA depletion. FIG. 5C shows uninduced χ8768-bHs (2.5×10⁶ CFU) was injected intravenously (i.v.) into NSG mice bearing orthotopic PANC-1 tumors (>250 mm³). After 48 hours, mice were then administered PBS (uninduced) or 250 mg L-arabinose (induced) by intraperitoneal injection. Tumors were isolated 16 hours later, sectioned and stained for ST and DAPI for subsequent immunofluorescence imaging. Tile-scanning was performed on entire tumor sections at 10× magnification. Areas of ST colonization under uninduced conditions are limited to small concentrated areas (arrows), whereas under induced conditions, greater areas of ST staining are observed (dotted lines), indicating greater diffusion caused by HA depletion. Representative tumors are shown for uninduced and induced groups. FIG. 5D demonstrates percent area of tumor colonized by χ8768-HAse under uninduced and induced conditions based on immunofluorescence. Percentage calculated using: (Area occupied by χ8768-Hase (green)/Total tumor area (DAPI))×100%. Areas (μm²) were determined using Image-Pro Plus (Media Cybernetics) analysis software. Error bars=standard error of the mean, *p<0.05, t-test. All data are representative of ≥3 experiments.

FIGS. 6A-6C demonstrate that ST-HAse potentiates the anti-tumor effects of gemcitabine treatment in PANC-1 tumor xenografts. FIG. 6A shows that s.c. tumors and skin (n=4) were isolated 3, 7 and 11 days post-induction (dpi), sectioned and stained for HA (red) and ST (green) for subsequent immunofluorescence (IF) imaging at 5× magnification. Trichrome staining of serial sections for same tissue sample also shown to left of IF images. Representative images shown. Arrows indicate area of ST/HA overlap. Scale bars=50 um. FIG. 6B shows that after 2 dpi, groups of tumor-bearing mice (n=6) were administered either gemcitabine (40 mg/kg) or diluent control (0.9% saline) by i.p. route, followed by additional administrations twice per week. PBS only group did not receive pre-treatment with ST-HAse. Tumors were measured weekly using a digital caliper. **p<0.01, ***p<0.001, ANOVA with Tukey's post hoc test. FIG. 6C shows that body weights were measured on indicated days following gemcitabine or control treatment and are presented as a percentage of initial body weight. n.s., not significant

FIGS. 7A-7E characterize bHs-expressing ST strains. FIG. 7A shows that BHs, expressed by induced ST strains, is not secreted into the culture media. LB culture media from induced bHs-expressing ST strains were run on a 4-20% polyacrylamide gradient gel and subjected to coomassie blue staining (CB) and western blot analysis against an amino terminal His-tag (α-His). Predicted bHs size ˜27 kDa (arrow). L=protein ladder. FIG. 7B shows the predicted bacterial subcellular localization of Streptomyces koganeinsis bHs using subcellular prediction software. Predicted (pred) locations—I: inside; i: inside tail; H: transmembrane helix; 0: outside; o: outside-tail. FIG. 7C are optical density readings (OD₆₀₀) for uninduced (solid blue circles) and induced (open red circles) χ8431-bHs. Cultures were done in triplicate and error bars represent standard error of the mean. FIG. 7D shows bacterial cells from uninduced (−) and induced (+2% L-arabinose) cultures of χ8429-bHs stained at indicated time points (4 and 24 hours) with acridine orange to indicate live bacterium and imaged by fluorescence microscopy at 63× magnification. Under induced conditions (+) the majority of χ8429-bHs at 4 and 24 hours post-induction are observed to have minimal (dim) staining (arrows), indicating dead bacterium. All data are representative of ≥3 experiments. In FIG. 7E, 1×10⁸ CFUs of χ8768-bHs were added to LB containing 0% or 2% L-arabinose and 0.4 mg/mL HA. The cetyltrimethylammonium bromide turbidimetric method (CTM) was used to determine rate of HA breakdown over 24 hours (OD₆₀₀). Error bars=standard error of the mean. All data are representative of ≥3 experiments.

FIG. 8 demonstrates that induced χ8768-bHs effectively depletes PC-3-derived hyaluronan. χ8768-bHs was grown for 3 hours in LB media containing 0% (uninduced) or 2% (induced) L-arabinose. 1×10⁸ CFUs were then co-incubated with HA^{high} human prostate cancer (PC-3) cells overnight. HA was detected using biotinylated HA-binding protein (HABP) followed by incubation with Vectastain strepavidin-HRP and ImmPACT DAB substrate. Specificity of HABP was confirmed through overnight incubation with 10 U/mL bovine hyaluronidase (Bov. Hs). Scale bar=100 uM. All images are representative of ≥3 experiments.

FIGS. 9A-9E demonstrate χ8768-bHs tumor colonization and vasculature in PANC-1 tumors. FIG. 9A shows that recombinant χ8768 transformed with a bacterial expression construct encoding the bioluminescent LUX cassette (χ8768-LUX) was injected intravenously (2.5×10⁶ colony forming units (CFU)) into NSG mice bearing orthotopic PANC-1 tumors (>250 mm³). LUX is constitutively expressed in viable bacteria. Mice were imaged on days 1, 3 and 5 by intravital bioluminescent imaging (representative mouse shown). FIG. 9B shows that PANC-1 tumor, spleen and liver were isolated from NSG mice 48 hours following intravenous injection of χ8768-LUX (2.5×10⁶ CFU) and imaged using a Lago X bioluminescent imager. FIG. 9C shows duct-like structures observed in PANC-1 sections of χ8768-bHs-treated mice, uninduced or induced. Representative fields are shown at 20× magnification. Scale bar=75 μm. Arrows indicate blood vessels. FIG. 9D are trichrome stained sections from PANC1 tumors containing induced or uninduced χ8768-bHs. Blood vessels are encircled by solid black lines. Sections serial to the trichrome slides were stained for ST showing representative patterns of concentration. ST staining under uninduced conditions are restricted to vessels (V), whereas ST staining under induced conditions can be observed outside of vessels (arrows), indicating enhance tumor permeability. FIG. 9E shows representative fields from trichrome stained slides that were counted for blood vessels and their diameters were measured in microns (m). Bar graph represents n>40 observations per slide. Error bars=standard error of the mean. ****p<0.0001, t-test. All data are representative of ≥3 experiments.

FIGS. 10A-10C demonstrate that induced χ8768-bHs causes no observable ST colonization or HA depletion in HA^{high} joints and does not decrease tumor cell density. Uninduced χ8768-bHs (2.5×10⁶ CFU) was injected i.v. into NSG mice bearing subcutaneous (s.c.) PANC-1 tumors (>150 mm³). After 48 hours, mice were then administered PBS (uninduced, U) or 240 mg L-arabinose (induced, I) by i.p. injection. In FIG. 10A, the joint between femur and tibia bones from hind leg (n=4) were isolated 3, 7 and 14 days post-i.p. injection (dpi), sectioned and stained for HA and ST for subsequent IF imaging at 5× magnification. Trichrome staining of serial sections for same joint shown to left of IF image. The overall amount of HA (arrows) is unchanged under induced conditions (compared to uninduced conditions), indicating that χ8768-bHs does not deplete HA in joints. Representative images shown. B, bone. Scale bars=100 μm. FIG. 10B are enlarged areas of serial tumor sections showing IF staining of HA and ST, trichrome (TC) and pan-cytokeratin (PC) in uninduced (U) and induced (I) conditions 3, 7 and 14 days post-i.p. injection (dpi). PC-staining is used to indicate areas of tumor cells. Regions enclosed by yellow rectangle represent magnified, high resolution images in FIG. 6A. Areas enclosed by dotted-lines are areas of PC-positive staining cells that are also colonized by χ8768-bHs (using corresponding IF imaging). Co-staining of PC markers with ST colonization indicates that ST are colonizing areas containing pancreatic tumor cells. Tile-scanning performed at 5× magnification. Scale bars=200 μm. FIG. 10C are serial sections of induced tumors (3 and 7 dpi) were stained for HA and nuclei for subsequent IF imaging at 5× magnification. Areas enclosed by dotted lines indicate areas that have been colonized by χ8768-bHs under induced conditions and have been depleted of HA as indicated by loss of HA staining (darker areas). Representative images shown. All images are representative of ≥2 experiments. Scale bars=50 μm.

FIGS. 11A-11C demonstrates expression, subcellular localization and toxicity of Streptomyces omiyaensis collagenase (CNase) expressed by attenuated VNP20009. FIG. 11A shows that attenuated VNP20009 transformed with the pBAD-CNase construct was cultured in Luria Broth (LB) containing 0% (uninduced) or 1% to 6% (induced) L-arabinose for up to 4 hours at 37° C. Bacterial cell lysates from 5×10⁷ colony forming units (CFUs) or 20× concentrated corresponding culture media at each time point for each L-arabinose concentration were run on a 4-20% polyacrylamide gradient gel and subjected to western blot analysis against a His-tag fused to the amino terminus of CNase (α-His). Predicted CNase size ˜31 kDa (arrow). In FIG. 11B, ST-CNase was cultured in LB media containing 1% L-arabinose for 1 hour and then immunostained (α-His) to determine subcellular localization of CNase. Expression of His-tagged CNase is only observed under induced conditions (arrows) and surrounding the cytoplasm. The cytosol (genomic DNA) is stained using DAPI. Representative images shown. Scale bar=1.0 μm. FIG. 11C is the growth curve of ST-CNase post-induction. Optical density readings (OD₆₀₀) for uninduced and induced (1% L-ara) ST-CNase cultures were measured over 24 hours. Uninduced and induced cultures were done in triplicate and error bars represent standard error of the mean. Growth curves of uninduced and induced are compared. For time points ≥2 hrs: **p<0.01, t-test.

FIGS. 12A-12D demonstrate hydrolytic collagenase activity by ST-CNase towards various substrates. FIG. 12A shows that uninduced or induced (1% L-arabinose) ST-CNase was incubated on LB-gelatin plates overnight (16 hr) at 37° C. Hydrolysis of gelatin in LB agar media is observed as opaque areas on LB-gelatin plates. Arrows indicate areas where uninduced or induced ST-CNase were spotted onto the plate. FIG. 12B-12D are hydrolysis reactions performed using uninduced or induced ST-CNase co-incubated with FITC-conjugated pig skin gelatin (FIG. 12B), bovine skin collagen type I (FIG. 12C) or human placenta collagen type IV (FIG. 12D) in 50 mM Tris-HCl (pH 8.0) containing 10 mM CaCl₂ at 37 C. Enzyme activity was measured by monitoring the fluorescence (FITC) (ex: 495 nm, em: 519 nm). Data are expressed as mean±SD of three independent experiments. *p<0.05, **p<0.01, p<0.001, t-test.

FIGS. 13A-13C demonstrate that in vivo depletion of collagenase by ST-CNase increases ST spread and is restricted to tumor tissue. FIG. 13A is immunofluorescence staining (IF) of ST-CNase in representative Pan02 tumors isolated from mice treated under uninduced (U) or induced (+L-arabinose, I) conditions with ST-CNase for 48 hours. In left panels, under induced conditions, ST-CNase can be observed to occupy greater areas of tumor (enclosed by dotted lines) compared to uninduced conditions. In right panels, magnified areas occupied by ST-CNase under induced conditions (enclosed by squares in IF) are also observed to be depleted of collagen (enclosed by squares in TC) as indicated by greater white space (loss of collagen) between tumor cells compared to uninduced conditions. FIG. 13B is the percent area of tumor colonized by ST-CNase under uninduced and induced conditions based on immunofluorescence. Percentage calculated using: (Area occupied by ST-CNase (green)/Total tumor area (DAPI))×100%. Areas (μm²) were determined using Image-Pro Plus (Media Cybernetics) analysis software. Error bars=standard error of the mean, *p<0.05, t-test. All data are representative of ≥2 experiments. FIG. 13C is TC staining of representative skin in Pan02-tumor bearing mice administered ST-CNase and then left uninduced or induced. Tissues examined in uninduced and induced conditions were isolated 72 hours post-induction. Data showed no ST colonization in skin and no loss of collagen content under induced conditions (compared to uninduced conditions). These results indicate that ST-CNase does not deplete collagen in collagen-high tissues.

### DETAILED DESCRIPTION

### I. Definitions

Before the present invention is further described, it is to be understood that this invention is not strictly limited to particular embodiments described, as such may of course vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

As used herein, the term “facultative anaerobe” refers to is an organism that makes ATP by aerobic respiration if oxygen is present, but is capable of switching to fermentation if oxygen is absent. Some examples of facultatively anaerobic bacteria are are Staphylococcus spp., Streptococcus spp. Escherichia coli, Salmonella, Listeria spp. and Shewanella oneidensis. Certain eukaryotes are also facultative anaerobes, including fungi such as Saccharomyces cerevisiae and many aquatic invertebrates such as Nereid (worm) polychaetes.

As used herein, the term “virulence” refers to a pathogen's or microbe's ability to infect or damage a host. In the context of animals, virulence refers to the degree of damage caused by a microbe to its host. The pathogenicity of an organism—its ability to cause disease—is determined by its virulence factors. The most commonly used measurement of virulence is the lethal dose required to kill 50% of infected hosts, referred to as the LD₅₀. The LD₅₀ measurement has the advantage that it allows comparisons across microbes, and the use of host death provides a nonequivocal endpoint. Some have developed approaches for measuring virulence that are not dependent on mortality.

As used herein, the term “attenuated” refers to a reduced virulence and to procedures that weaken an agent of disease (a pathogen). An attenuated pathogen is weakened, less vigorous compared to one that is non-attenuated. Attenuation may be due to genetic mutations. Genetic mutations may be engineered or result from passaging of the pathogen in cell culture. Au “attenuated facultative anaerobic bacterium,” as used herein, refers to a facultative anaerobic bacterium that has been altered to reduce virulence relative to the facultative anaerobic bacterium without the alteration, and is capable of replicating, in embodiments, the alteration is a genetic alteration of a gene that confers virulence to the unaltered facultative anaerobic bacterium.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

As used herein, the term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

As used herein, the term “operably linked” refers to a juxtaposition wherein the components described are in a relationship permitting them to function in their intended manner. In one embodiment, the term refers to a functional linkage between a nucleic acid expression control sequence (such as a promoter, and/or enhancer or other expression control sequence) and a second polynucleotide sequence, e.g., a polynucleotide-of-interest, wherein the expression control sequence directs transcription of the nucleic acid corresponding to the second sequence.

As used herein, the term “recombinant” generally refers to an organism, cell, or genetic material formed by recombination. Recombinant DNA (rDNA) molecules are DNA molecules formed by laboratory methods of genetic recombination (such as molecular cloning) to bring together genetic material from multiple sources, creating sequences that would not otherwise be found in the genome. Recombinant DNA is the general name for a piece of DNA that has been created by the combination of at least two strands. Recombinant DNA is possible because DNA molecules from all organisms share the same chemical structure, and differ only in the nucleotide sequence within that identical overall structure. The DNA sequences used in the construction of recombinant DNA molecules can originate from any species. For example, plant DNA may be joined to bacterial DNA, or human DNA may be joined with fungal DNA. In addition, DNA sequences that do not occur anywhere in nature may be created by the chemical synthesis of DNA, and incorporated into recombinant molecules. Using recombinant DNA technology and synthetic DNA, literally any DNA sequence may be created and introduced into any of a very wide range of living organisms. Recombinant DNA differs from genetic recombination in that the former results from artificial methods in the test tube, while the latter is a normal biological process that results in the remixing of existing DNA sequences in essentially all organisms.

As used herein, the term “recombinant proteins” refers to proteins that can result from the expression of recombinant DNA within living cells. When recombinant DNA encoding a protein is introduced into a host organism, the recombinant protein is not necessarily produced. Expression of foreign proteins requires the use of specialized expression vectors and often necessitates significant restructuring by foreign coding sequences.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “exogenous” refers to a molecule or substance (e.g., a compound, nucleic acid or protein) that originates from outside a given cell or organism. For example, an “exogenous promoter” as referred to herein is a promoter that does not originate from the organism it is expressed by. Conversely, the term “endogenous” or “endogenous promoter” refers to a molecule or substance that is native to, or originates within, a given cell or organism.

The term “promoter” as used herein refers to a recognition site of a polynucleotide (DNA or RNA) to which an RNA polymerase binds. The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA, which contains sequences capable of providing both promoter, and enhancer functions.

As used herein, the term “inducible promoter” refers to promoters can be regulated (induced) in presence of certain abiotic or biotic factors which may include certain biomolecules. These promoters are used by genetic engineers for regulating the expression of genes cloned in any organism by simply introducing the inducer. The two ways the activity of a promoter can be regulated are positive and negative control. Examples of inducible promoters include the pLac promoter, pTac promoter, a tetracycline-controlled promoter, and a pBAD promoter.

As used herein the term “hypoxia-inducible promoter” refers to promoters that are engineered to limit gene expression to hypoxic environments such as the tumor microenvironment. Examples of genes regulated by hypoxia-inducible promoters include pflE, hcp, menD, ansB, mltD, glpA, glpT, and pepT. Artificial promoters containing fumarate and nitrate reduction (FNR) regulator sites, such as FF+20* and hypoxia inducible promoter 1 (HIP1), are also examples of hypoxia-inducible promoters. See for example 61-64.

As used herein, the terms “polypeptide,” “peptide” and “protein” are used interchangeably and refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As used herein, the term “extracellular matrix” or “ECM” refers to a three-dimensional network of extracellular macromolecules, such as collagen, enzymes, and glycoproteins, that provide structural and biochemical support of surrounding cells. Because multicellularity evolved independently in different multicellular lineages, the composition of ECM varies between multicellular structures; however, cell adhesion, cell-to-cell communication and differentiation are common functions of the ECM. Components of the ECM are produced intracellularly by resident cells and secreted into the ECM via exocytosis. Once secreted, they then aggregate with the existing matrix. The ECM is composed of an interlocking mesh of fibrous proteins and glycosaminoglycans (GAGs). The extracellular matrix regulates tissue development and homeostasis, and its dysregulation contributes to neoplastic progression. In addition, a number of tumors and cancers overexpress components of the extracellular matrix, creating a fibrous barrier that prevents access by therapeutics to the tumor cells (See, for example, Refs. 75-79).

As used herein, the term “extracellular matrix degrading enzyme” refers to enzymes that degrade components of the extracellular matrix. Examples include but are not limited to matrix metalloproteinase, collagenase, hyaluronidase, chondroitinase, heparatinase, cathepsin, lyase, trypsin, protease, plasmin, and urokinase.

As used herein, the term “recombinant extracellular matrix degrading enzyme” refers to an extracellular matrix degrading enzyme produced by recombinant DNA and/or protein expression systems.

As used herein, the term “matrix metalloproteinase” or “MMP” or “matrixins”, are metalloproteinases that are calcium-dependent zinc-containing endopeptidases;’ other family members are adamalysins, serralysins, and astacins. The MMPs belong to a larger family of proteases known as the metzincin superfamily. Collectively, these enzymes are capable of degrading all kinds of extracellular matrix proteins, but also can process a number of bioactive molecules. MMPs are also thought to play a major role in cell behaviors such as cell proliferation, migration (adhesion/dispersion), differentiation, angiogenesis, apoptosis, and host defense.

As used herein, the term “collagenase” refers to enzymes that break the peptide bonds in collagen.

As used herein, the term “hyaluronidase” refers to a family of enzymes that catalyse the degradation of hyaluronic acid (HA).

As used herein, the term “chondroitinase” refers to enzymes that catalyse the degradation of chondroitin.

As used herein, the term “heparatinase” refers to enzymes that catalyze the degradation of heparin.

As used herein, the term “cathepsin” refers to a class of proteases (enzymes that degrade proteins) found in all animals as well as other organisms. There are approximately a dozen members of this family, which are distinguished by their structure, catalytic mechanism, and which proteins they cleave. Cathepsins have a vital role in mammalian cellular turnover.

As used herein, the term “lyase” an enzyme that catalyzes the breaking (an “elimination” reaction) of various chemical bonds by means other than hydrolysis (a “substitution” reaction) and oxidation, often forming a new double bond or a new ring structure.

As used herein, the term “trypsin” refers to s a serine protease from the PA clan superfamily, found in the digestive system of many vertebrates, where it hydrolyzes proteins. Trypsin is formed in the small intestine when its proenzyme form (trypsinogen produced by the pancreas) is activated. Trypsin cleaves peptide chains mainly at the carboxyl side of the amino acids lysine or arginine, except when either is followed by proline.

As used herein, the term “protease” refers to an enzyme that helps proteolysis which is protein catabolism by hydrolysis of peptide bonds. Proteases have evolved multiple times, and different classes of protease can perform the same reaction by completely different catalytic mechanisms. Proteases can be found in all forms of life and viruses.

As used herein, the term “plasmin” refers to a serine protease that degrades many blood plasma proteins, including fibrin clots. The degradation of fibrin is termed fibrinolysis, Apart from fibrinolysis, plasmin proteolyses proteins in various other systems: It activates collagenases, some mediators of the complement system, and weakens the wall of the Graafian follicle, leading to ovulation. It cleaves fibrin, fibronectin, thrombospondin, laminin, and von Willebrand factor.

As used herein, the term “urokinase” also known as” urokinase-type plasminogen activator” or “uPA” is a serine protease involved in degradation of the extracellular matrix and possibly tumor cell migration and proliferation.

The terms “disease” or “condition” refer to a state of being or health status of a patient or subject capable of being diagnosed and/or treated with compounds or methods provided herein. The disease may be a cancer.

As used herein, the term “cancer” refers to all types of cancer, neoplasm or malignant tumors found in mammals (e.g. humans). Examples of cancers that may be treated with a composition or method provided herein include solid tumors. In embodiments, the cancer is breast cancer, pancreatic cancer, or prostate cancer, or a subtype thereof. In embodiments, the pancreatic cancer is pancreatic ductal adenocarcinoma (PDAC).

“Treating” or “treatment” as used herein (and as well understood in the art) includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. “Treating” or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. In other words, “treatment” as used herein includes any cure, amelioration, or prevention of a disease.

“Treating” or “treatment” as used herein includes prophylactic treatment. Prophylactic treatment may inhibit the disease's spread; relieve the disease's symptoms, fully or partially remove the disease's underlying cause, shorten a disease's duration, or do a combination of these things. In embodiments, treating includes preventing. In embodiments, treating does not include preventing. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by diagnostic assays (e.g., assays described herein or known in the art). In some instances, chronic administration may be required. For example, the compositions are administered to the subject in an amount and for a duration sufficient to treat the patient.

The term “prevent” refers to a decrease in the occurrence of disease symptoms in a patient. The prevention may be complete (no detectable symptoms) or partial, such that fewer symptoms are observed than would likely occur absent treatment.

The term “patient” or “subject” refers to a living organism suffering from or prone to a disease or condition that can be treated by administration of a pharmaceutical composition. Non-limiting examples include humans, other mammals, bovines, rats, mice, dogs, monkeys, goat, sheep, cows, deer, and other non-mammalian animals. In some embodiments, a subject is human.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques.

The term “administering” as used herein refers to oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent. In embodiments, compositions described herein may be administered by intravenous, subcutaneous or intratumoral route.

The term “co-administer” as used herein refers to a composition described herein administered at the same time, just prior to, or just after the administration of one or more additional therapies. The compounds provided herein can be administered alone or can be coadministered to the patient. Co-administration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

The term “cancer model organism” as used herein refers to an organism exhibiting a phenotype indicative of cancer, or the activity of cancer causing elements, within the organism. A wide variety of organisms may serve as cancer model organisms, and include for example, cancer cells and mammalian organisms such as rodents (e.g. mouse or rat) and primates. Cancer cell lines are widely understood by those skilled in the art as cells exhibiting phenotypes or genotypes similar to in vivo cancers. Cancer cell lines as used herein includes cell lines from animals (e.g. mice) and from humans.

The term “anticancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rIL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

As used herein, the term “pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

Pharmaceutical compositions provided by the present invention include compositions wherein the active ingredient (e.g. compounds described herein, including embodiments or examples) may be contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., reducing, eliminating, or slowing the progression of disease symptoms. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

### II. Compositions

In an aspect, provided herein is an attenuated facultative anaerobic bacterium including a nucleic acid molecule encoding a recombinant extracellular matrix degrading enzyme operably linked to a promoter.

In embodiments, the attenuated facultative anaerobic bacterium is selected from Salmonella bongori, Salmonella choleraesuis, Salmonella enterica, Salmonella enteritidis, Salmonella paratyphi, Salmonella typhi, Salmonella typhimurium, Vibrio cholerae, Vibrio fischeri, Escherichia coli, Shigella boydii, Shigella dysenteriae, Shigella flexneri, Shigella sonnei, Lactobacillus bulgaricus, Listeria monocytogenes, Enterococcus faecalis, Enterococcus gallolyticus, Enterococcus faecium, and Streptococcus pyogenes. In embodiments, the attenuated facultative anaerobic bacterium is Salmonella bongori. In embodiments, the attenuated facultative anaerobic bacterium is Salmonella choleraesuis. In embodiments, the attenuated facultative anaerobic bacterium is Salmonella enterica. In embodiments, the attenuated facultative anaerobic bacterium is Salmonella enteritidis. In embodiments, the attenuated facultative anaerobic bacterium is Salmonella paratyphi. In embodiments, the attenuated facultative anaerobic bacterium is Salmonella typhi. In embodiments, the attenuated facultative anaerobic bacterium is Salmonella typhimurium. In embodiments, the attenuated facultative anaerobic bacterium is Vibrio cholera. In embodiments, the attenuated facultative anaerobic bacterium is Vibrio fischeri. In embodiments, the attenuated facultative anaerobic bacterium is Escherichia coli. In embodiments, the attenuated facultative anaerobic bacterium is Shigella boydii. In embodiments, the attenuated facultative anaerobic bacterium is Shigella dysenteriae. In embodiments, the attenuated facultative anaerobic bacterium is Shigella flexneri. In embodiments, the attenuated facultative anaerobic bacterium is Shigella sonnei. In embodiments, the attenuated facultative anaerobic bacterium is Lactobacillus bulgaricus. In embodiments, the attenuated facultative anaerobic bacterium is Listeria monocytogenes. In embodiments, the attenuated facultative anaerobic bacterium is Enterococcus faecalis. In embodiments, the attenuated facultative anaerobic bacterium is Enterococcus gallolyticus. In embodiments, the attenuated facultative anaerobic bacterium is Enterococcus faecium. In embodiments, the attenuated facultative anaerobic bacterium is Streptococcus pyogenes.

In embodiments, the attenuated facultative anaerobic bacterium is a Salmonella typhimurium strain selected from MVP728 (see, for example, Ref 65), YS1646 (VNP20009) (see, for example, Refs. 66-67), RE88 (see, for example, Ref 68), LH430 (see, for example, Ref. 69), SL7207 (see, for example, Refs. 70-71), χ8429, χ8431 and χ8768 (see, for example, Refs. 72-73). In embodiments, the attenuated facultative anaerobic bacterium is Salmonella typhimurium MVP728. Y In embodiments, the attenuated facultative anaerobic bacterium is Salmonella typhimurium S1646 (VNP20009). In embodiments, the attenuated facultative anaerobic bacterium is Salmonella typhimurium RE88. In embodiments, the attenuated facultative anaerobic bacterium is Salmonella typhimurium LH430. In embodiments, the attenuated facultative anaerobic bacterium is Salmonella typhimurium S L7207. In embodiments, the attenuated facultative anaerobic bacterium is Salmonella typhimurium χ8429. In embodiments, the attenuated facultative anaerobic bacterium is Salmonella typhimurium χ8431. In embodiments, the attenuated facultative anaerobic bacterium is Salmonella typhimurium χ8′768. (See for example Bollen et al. 2008; Rodriguez et al. 2012)

In embodiments, the attenuated bacterium is attenuated by alteration or mutation of genes. Attenuation includes genetic mutations that prevent expression of virulence-associated genes. Genes include those that encode for proteins contributing to recombination (recA), nucleotide synthesis (purl), motility (fliC), cell wall composition (msbB, asd), transcription regulation (phoP, phoQ) and amino acid synthesis (aroA). In embodiments, attenuated bacterium have one or more gene mutations. In embodiments, attenuated bacterium have one or more mutations in a gene selected from recA, purl, fliC, msbB, asd, phoP, phoQ, and aroA (see, for example, Ref 74).

In embodiments, the nucleic acid molecule is an expression vector or plasmid. In embodiments, the nucleic acid molecule includes elements for gene transcription and translation. In embodiments, the elements for gene transcription and translation include an origin of replication, a selectable marker, a gene, and a promoter. In embodiments, the gene is transcribed and translated to produce a functional protein or gene product.

In embodiments, the nucleic acid molecule includes a gene encoding a recombinant extracellular matrix degrading enzyme. In embodiments, the gene is transcribed and translated to produce a functional extracellular matrix degrading enzyme. In embodiments, the extracellular matrix degrading enzyme is a bacterial extracellular matrix degrading enzyme. In embodiments, the extracellular matrix degrading enzyme is a human extracellular matrix degrading enzyme In embodiments, the extracellular matrix degrading enzyme is a parasitic extracellular matrix degrading enzyme.

In embodiments, the recombinant extracellular matrix degrading enzyme is selected from matrix metalloproteinase, collagenase, hyaluronidase, chondroitinase, heparatinase, cathepsin, lyase, trypsin, protease, plasmin, and urokinase. In embodiments, the recombinant extracellular matrix degrading enzyme is matrix metalloproteinase. In embodiments, the recombinant extracellular matrix degrading enzyme is collagenase. In embodiments, the recombinant extracellular matrix degrading enzyme is hyaluronidase. In embodiments, the recombinant extracellular matrix degrading enzyme is chondroitinase. In embodiments, the recombinant extracellular matrix degrading enzyme is heparatinase. In embodiments, the recombinant extracellular matrix degrading enzyme is cathepsin. In embodiments, the recombinant extracellular matrix degrading enzyme is lyase. In embodiments, the recombinant extracellular matrix degrading enzyme is trypsin. In embodiments, the recombinant extracellular matrix degrading enzyme is protease. In embodiments, the recombinant extracellular matrix degrading enzyme is plasmin. In embodiments, the recombinant extracellular matrix degrading enzyme is urokinase. In embodiments, the recombinant extracellular matrix degrading enzyme is a protease encoded by a codon optimized nucleic acid comprising SEQ ID NO.: 2. In embodiments, the recombinant extracellular matrix degrading enzyme is a Streptomyces omiyaensis trypsin-like protease encoded by a codon optimized nucleic acid comprising SEQ ID NO.: 2.

In embodiments, the hyaluronidase is bacterial hyaluronidase. In embodiments, the bacterial hyaluronidase is a selected from Streptomyces koganeiensis, Streptomyces hyaluronlyticus, Staphylococcus aureus, Streptococcus pyogenes and Clostridium perfringens. In embodiments, the bacterial hyaluronidase is from Streptomyces koganeiensis. In embodiments, the bacterial hyaluronidase is from Streptomyces hyaluronlyticus. In embodiments, the bacterial hyaluronidase is from Staphylococcus aureus. In embodiments, the bacterial hyaluronidase is from Streptococcus pyogenes. In embodiments, the bacterial hyaluronidase is from Clostridium perfringens. In embodiments, the bacterial hyaluronidase is encoded by a codon optimized nucleic acid sequence comprising SEQ ID NO.: 1. In embodiments, the bacterial hyaluronidase is a Streptomyces koganeinsis hyaluronidase encoded by a codon optimized nucleic acid sequence comprising SEQ ID NO.: 1.

In embodiments, the promoter is an inducible promoter. In embodiments, the inducible promoter is selected from a pLac promoter, pTac promoter, a tetracycline-controlled promoter, and a pBAD promoter. In embodiments, the inducible promoter is a pLac promoter. In embodiments, the inducible promoter is a pTac promoter. In embodiments, the inducible promoter is a tetracycline-controlled promoter. In embodiments, the inducible promoter is a pBAD promoter.

In embodiments, the promoter is a tumor-specific promoter.

In embodiments, the promoter is a hypoxia-inducible bacterial promoter. In embodiments, the hypoxia-inducible bacterial promoter is selected from those regulating expression of spflE, hcp, menD, ansB, mltD, glpA, glpT, and pepT. In embodiments, the hypoxia-inducible bacterial promoter is a spflE promoter. In embodiments, the hypoxia-inducible bacterial promoter is a hcp promoter. In embodiments, the hypoxia-inducible bacterial promoter is a mend promoter. In embodiments, the hypoxia-inducible bacterial promoter is an ansB promoter. In embodiments, the hypoxia-inducible bacterial promoter is a mltD promoter. In embodiments, the hypoxia-inducible bacterial promoter is a glpA promoter. In embodiments, the hypoxia-inducible bacterial promoter is a glpT promoter. In embodiments, the hypoxia-inducible bacterial promoter is a pepT promoter. In embodiments, the hypoxia-inducible bacterial promoter is a FF+20* promoter. In embodiments, the hypoxia-inducible bacterial promoter is a HIP1 promoter.

In embodiments, the attenuated facultative anaerobic bacterium expresses an extracellular matrix degrading enzyme under tumor-specific conditions such as hypoxia.

### III. Methods of Use

In an aspect, provided herein is a method of treating a tumor in a subject including administering to the subject an effective amount of an attenuated facultative anaerobic bacterium that includes a nucleic acid molecule encoding a recombinant extracellular matrix degrading enzyme operably linked to a promoter.

In an aspect, provided herein is a method of treating a tumor in a subject including administering to the subject an effective amount of an attenuated facultative anaerobic bacterium and a chemotherapeutic agent. The attenuated facultative anaerobic bacterium includes a nucleic acid molecule encoding a recombinant extracellular matrix degrading enzyme operably linked to a promoter.

In embodiments, the tumor is a solid tumor. In embodiments, the tumor is selected from pancreatic, breast, prostate, skin, lung, and abdomen tumor. In embodiments, the tumor is a pancreatic tumor. In embodiments, the tumor is a breast tumor. In embodiments, the tumor is a prostate tumor. In embodiments, the tumor is a skin tumor. In embodiments, the tumor is lung tumor. In embodiments, the tumor is an abdomen tumor.

In embodiments, the pancreatic tumor is pancreatic ductal adenocarcinoma. In embodiments, the skin tumor is malignant melanoma. In embodiments, the skin tumor is desmoplastic squamous cell carcinoma. In embodiments, the lung tumor is small cell lung cancer. In embodiments, the lung tumor is non-small cell lung cancer. In embodiments, the abdomen tumor is desmoplastic small round cell tumor.

In embodiments, the methods provided herein include administering to a subject an effective amount of attenuated facultative anaerobic bacterium. In embodiments, compositions described herein may be administered by intravenous, intraperitoneal, subcutaneous or intratumoral route.

In embodiments, an effective amount of an attenuated facultative anaerobic bacterium is an amount such that expression of the extracellular matrix degrading enzyme is sufficient to degrade the extracellular matrix, degrade a component of the extracellular matrix, and/or increase penetration of an anti-tumor agent to the tumor. In embodiments, an effective amount of an attenuated facultative anaerobic bacterium is an amount such that expression of the extracellular matrix degrading enzyme is sufficient to degrade the extracellular matrix. In embodiments, an effective amount of an attenuated facultative anaerobic bacterium is an amount such that expression of the extracellular matrix degrading enzyme is sufficient to degrade a component of the extracellular matrix. In embodiments, an effective amount of an attenuated facultative anaerobic bacterium is an amount such that expression of the extracellular matrix degrading enzyme is sufficient to increase penetration of an anti-tumor agent to the tumor.

In embodiments, the attenuated facultative anaerobic bacterium includes a nucleic acid molecule encoding a recombinant extracellular matrix degrading enzyme operably linked to a promoter as described above.

In embodiments, administration of the attenuated facultative anaerobic bacterium that includes a nucleic acid molecule encoding a recombinant extracellular matrix degrading enzyme operably linked to a promoter as described leads to expression of extracellular matrix degrading enzyme capable of degrading components of the extracellular matrix. Degradation of components of the extracellular matrix leads to a weakness in the fibrous surroundings of the tumor and thus, access to the tumor by chemotherapeutic agents.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Development of Attenuated ST Strains Expressing bHs from S. koganeiensis

Various attenuated Salmonella typhi (ST) strains expressing bacterial hyaluronidase (bHs) from S. koganeiensis and referred to as bHs-ST have been developed and characterized. The data demonstrated that attenuated ST is capable of auto-displaying functional bHs that can effectively degrade purified and tumor-derived hyaluron (HA). The data confirmed bHs-ST, when delivered systemically, is capable of colonizing orthotopic human pancreatic adenocarcinoma (PDAC) tumors in mice and can cause remarkable degradation of tumor-derived HA resulting in enhanced diffusion of ST throughout the tumor. This was the first example of a tumor-specific, extracellular matrix (ECM)-degrading strategy that could significantly improve efficacy of therapies for PDAC and other desmoplastic tumor types.

Materials and Methods

Animals and Cell Lines

Non SCID gamma (NSG) mice were obtained from breeding colonies housed at the City of Hope (COH) Animal Research Center and, for all studies, handled according to standard IACUC guidelines (approved IACUC protocol 17128). The PANC-1 and PC-3 cell lines were obtained from ATCC® (CRL1469™, CRL1435™). PC-3 cells were maintained in RPMI media containing 10% FBS, 2 mM L-glutamine and pen/strep. PANC-1 cells, prior to orthotopic implantation (survival surgery) into NSG mice, were passaged ≤5 times and maintained at ≤80% confluency in DMEM containing 10% FBS, 2 mM L-glutamine and pen/strep.

ST Strains and Generation of bHs-ST

YS1646 was obtained from ATCC® (202165™). Other attenuated strains were kind gifts obtained from Roy Curtiss III (χ8429, χ8431, χ8768), B. A. D Stocker (SL7207) and Michael Hensel (MVP728) (35-39). YS1646 was cultured in modified LB media containing MgSO₄ and CaCl₂ place of NaCl. All other strains were cultured in Miller LB media (Fisher BioReagents). The S. koganeinsis bHs amino acid sequence (GenBank Accession no. KP313606) was used to synthesize a codon-optimized cDNA (Biomatik) inserted in-frame into a 6×His-pBAD bacterial expression vector (kind gift from Michael Davidson, Addgene #54762) using XhoI/EcoRI sites. In-frame insertion of bHs into the pBAD vector adds a 6×His tag to the N-terminus of the protein. χ8768-LUX was generated using the pAKlux2 plasmid (pAKlux2 was a gift from Attila Karsi, Addgene #14080). Plasmids were electroporated into ST strains using a BTX electroporator (1 mm gap cuvettes, settings: 1.8 kV, 186 ohms), spread onto LB plates containing 100 ug/mL ampicillin and incubated overnight at 37° C. Glycerol stocks were generated for pBAD-bHs-positive clones identified by colony PCR and restriction digest of plasmid preparations.

Bacterial Growth, Viability and Analysis of bHs Expression

ST clones electroporated with pBAD-bHs were cultured in media with or without 2% (w/v) L-arabinose at 37° C., 225 rpm for time intervals ranging from 3 hr-24 hr. Growth kinetics were monitored through absorbance readings at 600 nm (Genesys 30, Thermo Scientific) every 1-2 hrs, up to 24 hrs. 6×His-tagged bHs expression was detected in bacterial lysates by western blot and localization of bHs was detected by immunofluorescence using a primary monoclonal mouse anti-6×His antibody (Proteintech). For immunofluorescence, uninduced and induced ST grown for ˜3 hours were fixed with 4% paraformaldehyde at room temperature (RT) for 30 minutes, and permeabilized with 0.1% Triton-X 100/PBS pH=7.2 at RT for 30 minutes followed by lysozyme (Sigma, 100 ug/mL final concentration in 5 mM EDTA) at RT for 45 minutes. Fixed/permeabilized bacteria were incubated with primary antibody (1:100) for 30 minutes with shaking in a humidified 37° C. incubator followed by incubation with FITC-conjugated anti-mouse secondary (1:200, Abcam) and DAPI for 30 minutes with shaking in a humidified 37° C. incubator.

Hyaluronan-BSA LB (HBL) Plate and Turbidimetric Assays

HBL plates for evaluating hyaluronidase activity were generated as previously described (40). Briefly, LB agar plates containing final concentrations of 0.4 mg/mL HA (Sigma, H-1504), 1% bovine serum albumin fraction V (Sigma) and 100 ug/mL ampicillin (Sigma) were used for plating uninduced and induced ST strains (10₆ colony forming units (CFU)/5 uL drop) at 37° C. for 16-24 hrs. Plates were then flooded with 2N glacial acetic acid. Clear zones were observed against a background of opaque precipitated BSA conjugated to the undigested HA. For turbidimetric quantification of HA degradation in culture media over time, the cetyltrimethylammonium bromide (CTAB) turbidimetric method was used (41). In brief, LB media containing 0.4 mg/mL HA and 100 ug/mL ampicillin, with or without 2% L-arabinose, was used to culture bHs-ST strains (2 mL starting volume) over 24 hrs at 37° C., 225 rpm. HA content (absorbance) in culture media (100 uL aliquot) was measured every 2-3 hrs by addition of 2.5% CTAB reagent (25 uL, Sigma) and absorbance read at 600 nm.

Orthotopic Tumor Implantation

Previously published methods were used for survival surgery and orthotopic implantation of PANC-1 cells into the pancreas of NSG mice (42). Briefly, while anesthetized and using sterile techniques, a small incision was made in the skin and peritoneal lining and the pancreas externalized. Using a 27 gauge needle, approximately 1.5-2×10⁶ PANC-1 cells in a volume of 50 uL Matrigel (BD Biosciences) were injected into the body of the pancreas. The pancreas was then reinserted into the peritoneal space and inner and outer incisions were closed using absorbable sutures and staples, respectively. Analgesics were administered pre- and post-surgery.

Immunohistochemistry/Immunofluorescence (IHC/IFC) to Detect HA and ST

Prior to incubation with bHs-ST in vitro, sections of PANC-1 tumors were de-paraffinized and rehydrated. Uninduced and induced χ8768-bHs (10⁸ CFU), PBS or bovine hyaluronidase (Sigma) were incubated on tissue sections overnight in a humidified 37° C. incubator. Following treatment, specimens were incubated with a biotinylated HA binding protein (HABP, Sigma) at 5 ug/mL final concentration for 2 hours at 37° C. Slides were then washed, incubated with streptavidin-HRP at RT for 1 hr and visualized with a DAB kit (Vectastain). Hematoxylin was used for counterstaining.

PANC-1 tumor sections from NSG mice treated intravenously with χ8768-bHs and then induced, or left uninduced, were de-paraffinized and rehydrated and stained overnight with 2.5 ug/mL HABP, 1:100 anti-ST antibody (Santa Cruz, sc-52223), or according to Masson's Trichrome protocol by the Pathology Research Services Core (City of Hope). Strepavidin-PE and anti-mouse-Cy5 were then used to visualize HA and ST by fluorescence microscopy (Zeiss Observer II), in addition to DAPI for visualizing nuclei. Tiling was performed at 10×, while high magnification to visualize ST was done at 100× (oil).

Administration and Induction of χ8768-bHs in PANC-1 Tumor-Bearing NSG Mice

NSG mice with palpable PANC-1 tumors (>250 mm₃) were intravenously injected with 2.5×10⁶ χ8768-LUX or χ8768-bHs. Actively growing χ8768-LUX is constitutively bioluminescent and was used to evaluate χ8768 colonization of PANC-1 tumors in vivo using intravital imaging (LagoX, Spectral Imaging). Two days after administrating χ8768-bHs, mice were administered 240 mg L-arabinose or PBS (intraperitoneally). Mice were euthanized 16 hours after receiving L-arabinose or PBS and tumors were excised, fixed and sectioned to evaluate HA and ST.

Blood Vessel Dilation Measurements

Ten to twelve fields at 10× for Trichrome stained slides from PANC1 tumors treated with χ8768-bHs and induced with L-arabinose or uninduced in vivo were imaged using a Leica DMi8 Microscope. In each field, blood vessel diameters were measured using the Leica LasX software. A Mann-Whitney test was performed on values using the Prism 7.2 software from GraphPad.

Tightly-Regulated Expression of bHs by Attenuated ST Strains

In order to circumvent potential toxic effects of constitutive bHs expression on attenuated ST strains, a tightly-regulated inducible expression system was employed. Inducible expression in ST is possible through the use of a construct containing the PBAD promoter of the araBAD (arabinose) operon and the gene encoding the positive and negative regulator of this promoter, araC (for example, ref. 43). An ST codon-optimized bHs sequence, based on the amino acid sequence of the well-characterized S. koganeiensis bHs, was synthesized and cloned into a previously described pBAD vector to generate pBAD-bHs (44). A single plasmid preparation of pBAD-bHs was used for electroporation into various attenuated strains of ST (Table 1).

TABLE 1
ST strains used in these studies.

| | | Parental (wildtype) | |
|---|---|---|---|
| ST Strain | Mutations | ST strain | Referene(s) |
| SL7207 | aroA⁻ | SL1344 | (37, 39) |
| MVP728 | purD⁻/htrA⁻ | ATCC14028 | (36) |
| YS1646 | msbB⁻/purI⁻ | ATCC14028 | (30) |
| _{χ}8429 | ΔphoP/phoQ | UK-1 | (38) |
| _{χ}8431 | ΔphoP/phoQ | UK-1 | (38) |
| _{χ}8768 | ΔphoP233 | UK-1 | (35) |

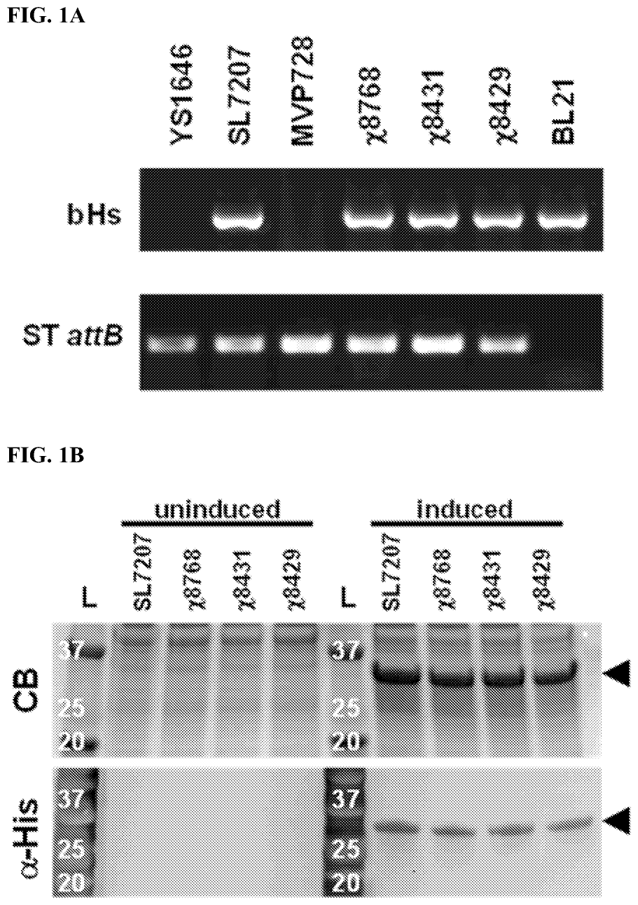

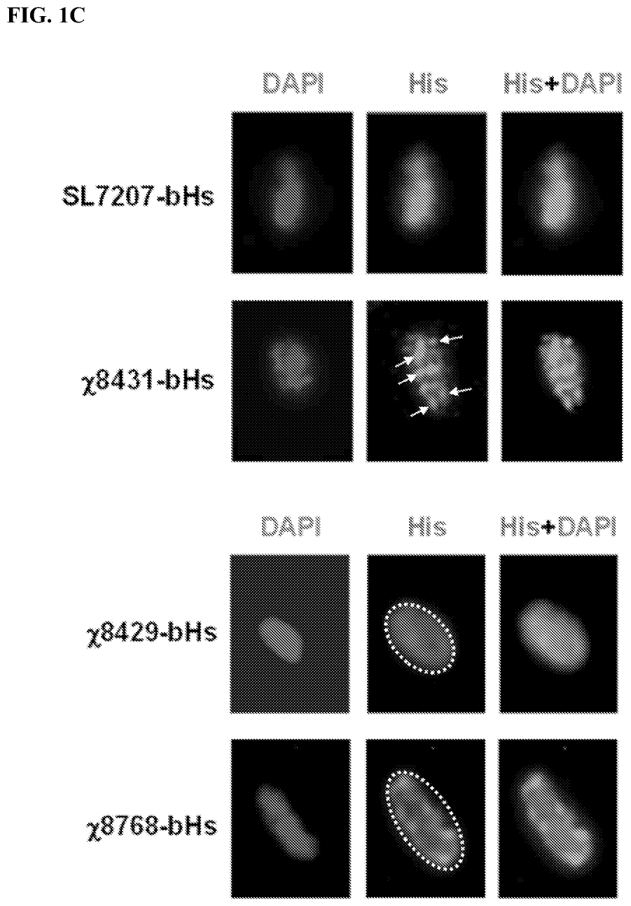

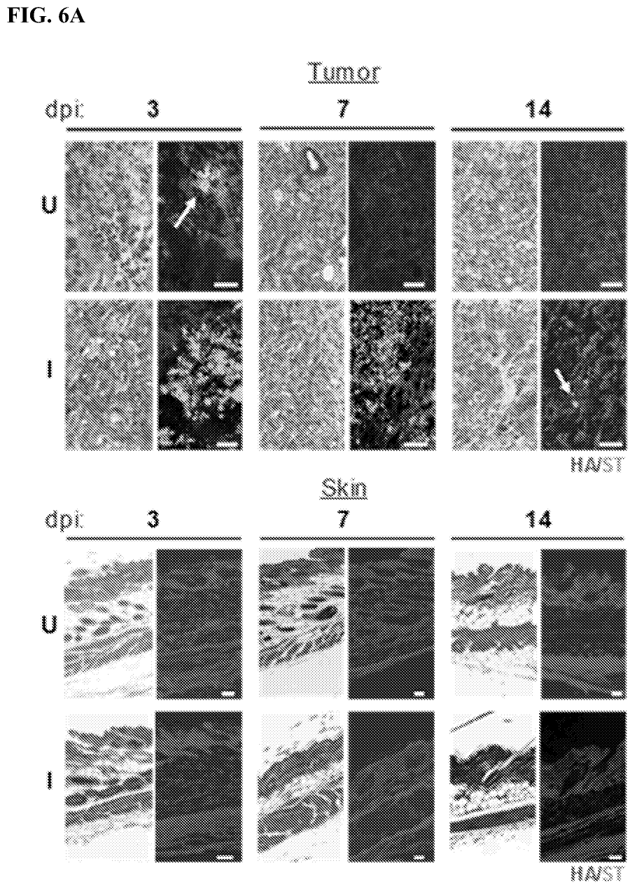

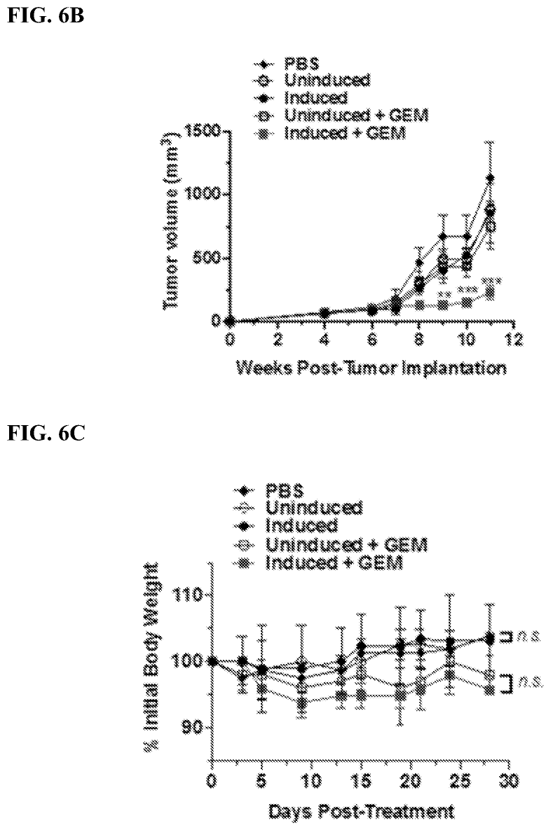

Colony polymerase chain reaction (PCR) was performed for each transformed strain (≥8 colonies) to detect for retention of the bHs transgene (FIG. 1A). All ampicillin-resistant colonies examined for YS1646 and MVP728 were completely negative for the bHs transgene in the absence of L-arabinose, suggesting loss of the transgene independent of induced protein expression. Of note, both YS1646 and MVP728 are derived from the same parental strain ATCC 14028. Culturing of pBAD-bHs-positive colonies in uninduced and induced (+2% L-arabinose) conditions, followed by coomassie blue (CB) staining and western blot (WB) of pellet lysates, revealed expression of His-tagged bHs at the correct molecular weight (27 kD) as well as tight regulation of protein expression (FIG. 1B). No bHs was detected in culture media by CB or WB (FIG. 6A), suggesting that bHs is not secreted by these ST strains following induction.

Using HHMTOP, PSORTb and CellP-Loc subcellular localization prediction tools, bHs is predicted to be anchored to the cytoplasmic membrane at its N-terminus, while the active region (residues 66-247) is localized to the outer membrane/extracellular space (FIG. 6B) (For example, Refs. 45-47). To determine the subcellular location of bHs expressed by the various ST strains, immunofluorescence staining was performed utilizing a 6×His-tag fused to the N-terminus of the bHs protein was (FIG. 1C). His-tagged bHs expressed by induced χ8429-bHs and χ8768-bHs would reveal clear bHs localization outside of the bacterial cytoplasm, defined by DAPI staining of genomic DNA. In contrast, bHs expressed by SL7207-bHs, and to a smaller extent in χ8431-bHs, is localized to the cytoplasm, suggesting impaired transport and formation of inclusion bodies in these attenuated strains. No detectable bHs expression was observed in uninduced cultures (data not shown). Altogether, these data confirm that expression of the ST codon-optimized bHs transgene is tightly regulated using an inducible pBAD system and that the expressed bHs protein can be autodisplayed on the bacterial surface.

Growth Kinetics and Viability of bHs-Expressing ST Strains

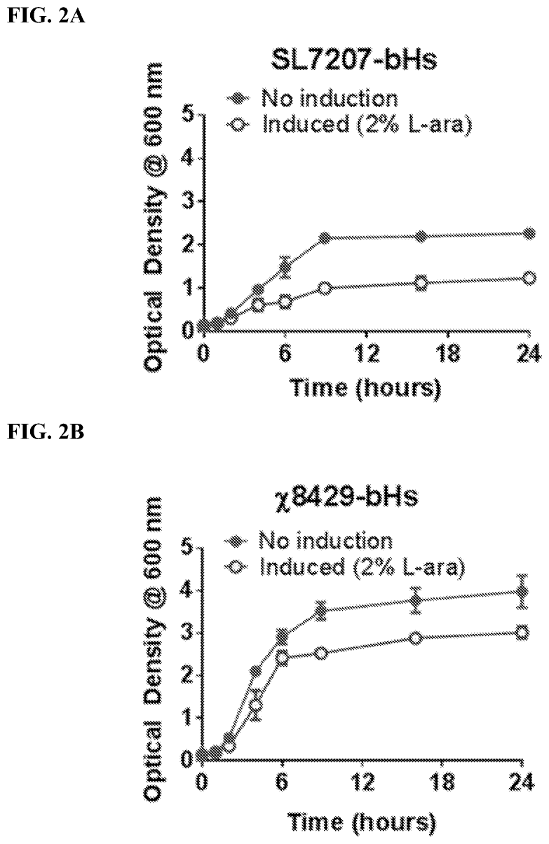

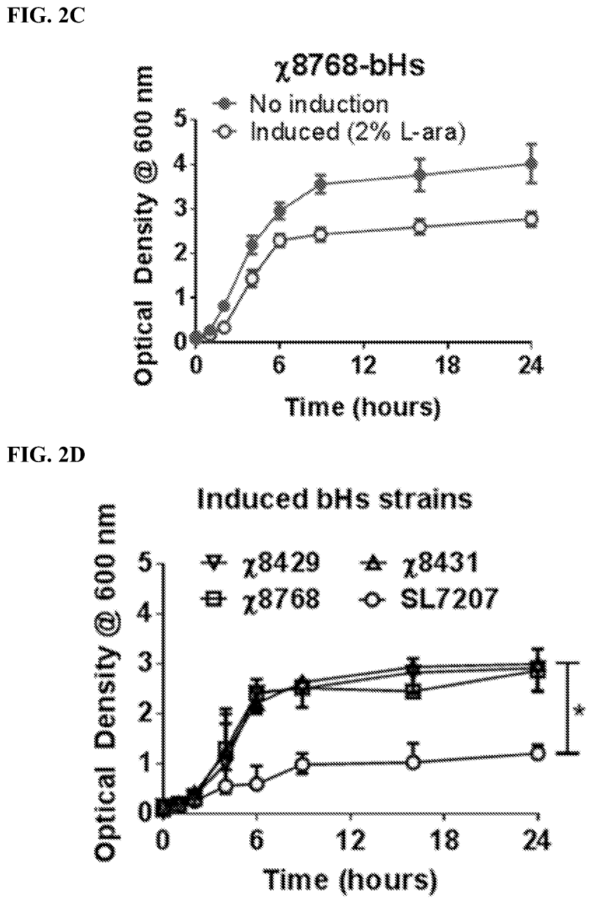

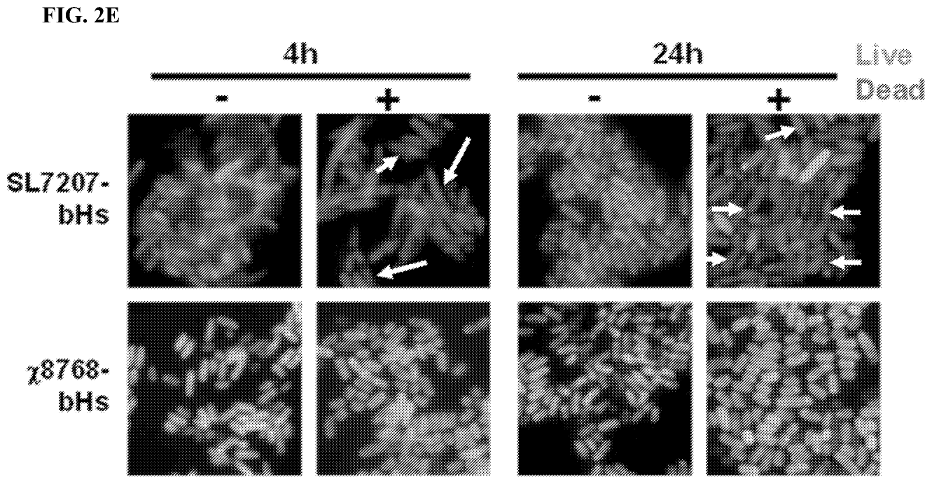

While tight regulation of transgene expression is important for minimizing toxicity during initial growth stages, sufficient growth and viability following induction will also be critical to maximizing bHs activity. Thus, growth kinetics of each of the bHs-ST-expressing strains over 24 hours in non-inducing and inducing (±2% L-arabinose) conditions was determined. SL7207 alone is already known to have dramatically reduced growth kinetics compared to wildtype ST, reaching a maximum optical density (O.D.) 2-3 fold lower than other attenuated strains (FIG. 2). Under induced conditions, the maximum O.D. for SL7207-bHs is significantly reduced to under 1 (FIG. 2A), whereas other attenuated ST strains could reach maximum O.D.s 3-fold higher (FIG. 2B-D). Both inherently poor growth kinetics of unmodified SL7207 as well as cytoplasmic accumulation of bHs (FIG. 1C), could contribute to the significantly reduced growth kinetics of SL7207-bHs following induction. Interestingly, while χ8431-bHs showed mixed localization of bHs by immunofluorescence, induced growth kinetics were indistinguishable from χ8768-bHs and χ8429-bHs. These data suggest that χ8768, χ8431 and χ8429 have greater viability upon bHs induction, which could potentially translate into more extensive HA degradation.

To further investigate bacterial viability after induction, live/dead staining was performed using a mixture of acridine orange (AO) and ethidium bromide (EB), respectively, during log phase (4 hr) and stationary phase (24 hr) of uninduced and induced cultures (For example 48, 49). As shown in FIG. 2E and FIG. 6C, the percentage of viable bacterial cells after induction of SL7207-bHs is significantly lower than χ8768- and χ8429-bHs, as indicated by highly EB-positive SL7207-HAse as early as 4 hr and continuing 24 hr after induction. These results further emphasize the deleterious toxic effects of bHs expression on the viability of attenuated strains such as SL7207 but also highlight ST strains capable of autodisplaying bHs and remaining viable during and long after initiation of induction.

BHs-ST Strains Degrade Purified HA

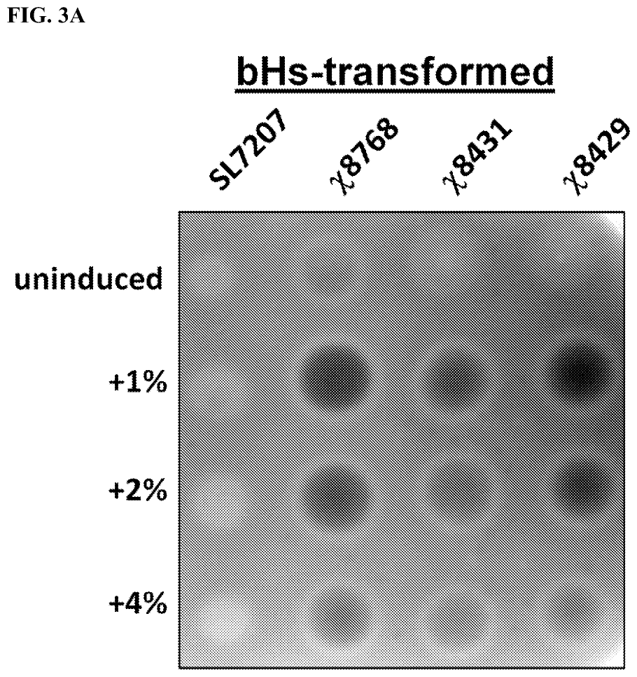

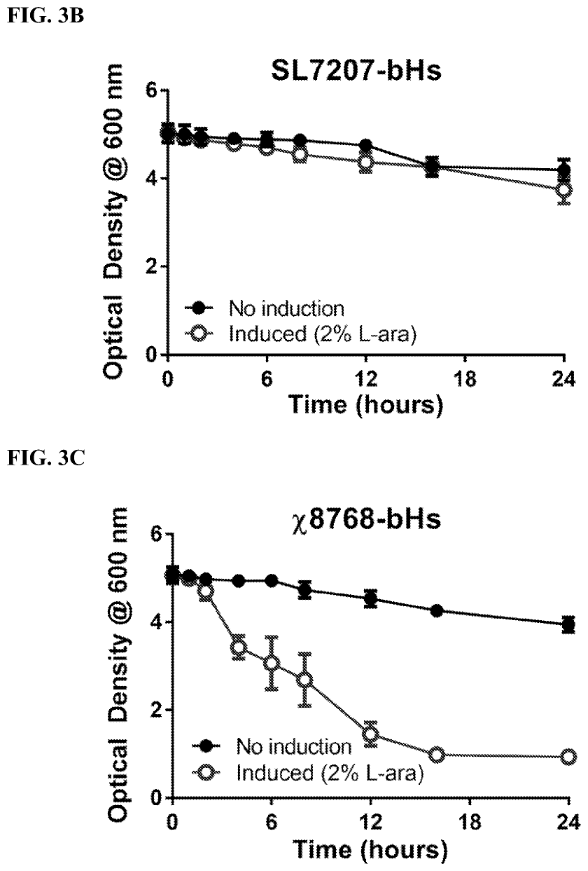

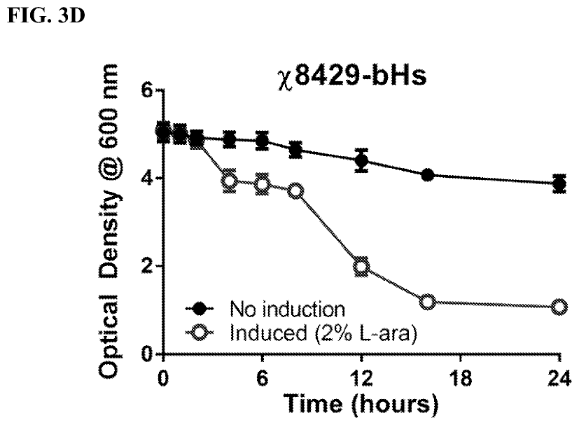

To test the functionality of bHs expressed by the various attenuated ST strains, HA agar plate clearing and liquid culture turbidimetric assays (for example 40, 41) were employed. For plate clearing assays, HA and BSA are mixed into LB agar plates (HBL plates). Addition of 2N acetic acid to HBL plates containing intact HA will form a white precipitate with BSA, while areas of HA degradation will remain clear. BHs-expressing strains were pre-induced for 3 hours in LB media containing 0 to 4% L-arabinose and then spotted (1×10₈ CFU/5 uL) onto HBL plates overnight. After flooding HBL plates with 2N acetic acid, zones of clearing were observed for χ8768-, χ8431- and χ8429-bHs, but not SL7207-bHs (FIG. 3A). Interestingly, χ8431-bHs, which had exhibited intermediate surface display of bHs (FIG. 1C), also demonstrated intermediate HA degradation. These data suggest that ST strains that efficiently display bHs on their surface and exhibit greater viability are far more effective at degrading pure HA.

To determine the kinetics of HA degradation by the various bHs-ST strains, each was cultured under uninduced and induced conditions in LB media containing HA (0.4 mg/mL) and measured HA content over a 24 hour period using the cetyltrimethylammonium bromide (CTAB) turbidimetric method (39). At each time point, high molecular weight HA in culture media was precipitated with CTAB and optical density determined at a wavelength of 600 nm (FIG. 3B-D). Data showed higher overall rates of HA degradation over the 24 hr period by χ8′768- and χ8429-bHs (˜0.15 O.D. units/hr), an intermediate rate for χ8431-bHs (˜0.10 O.D. units/hr) and no degradation by SL7207-bHs, which recapitulates activity observed for each strain on HBL plates. The highest rate of degradation was observed for χ8768-bHs during the first 12 hours of induction (˜0.3 O.D. units/hr), whereas χ8429-bHs and χ8431-bHS showed two times less activity (˜0.15 O.D. units/hr). Overall, these data indicate that the χ8768-bHS strain is most effective in degrading purified HA within hours of induction.

χ8768-bHs Effectively Degrades Tumor-Derived HA

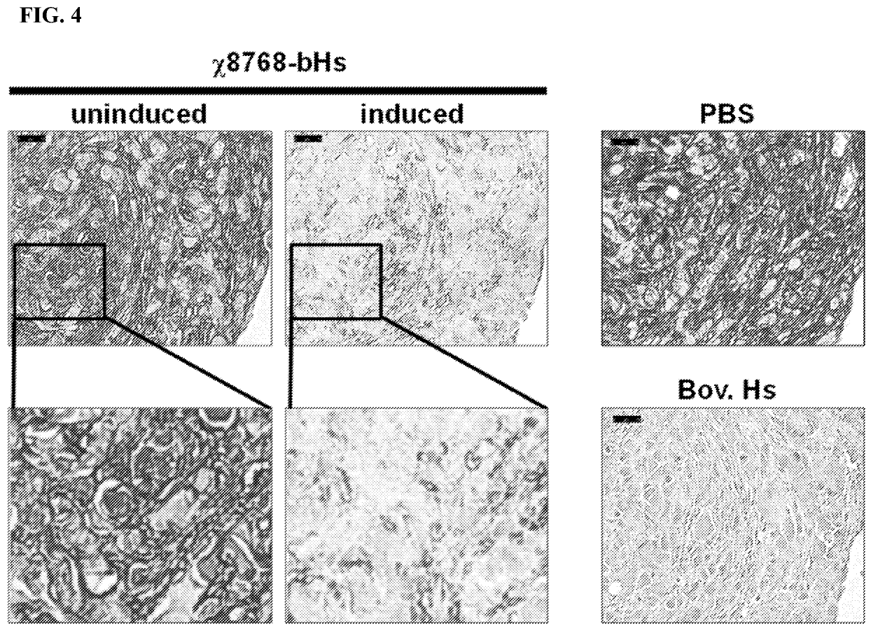

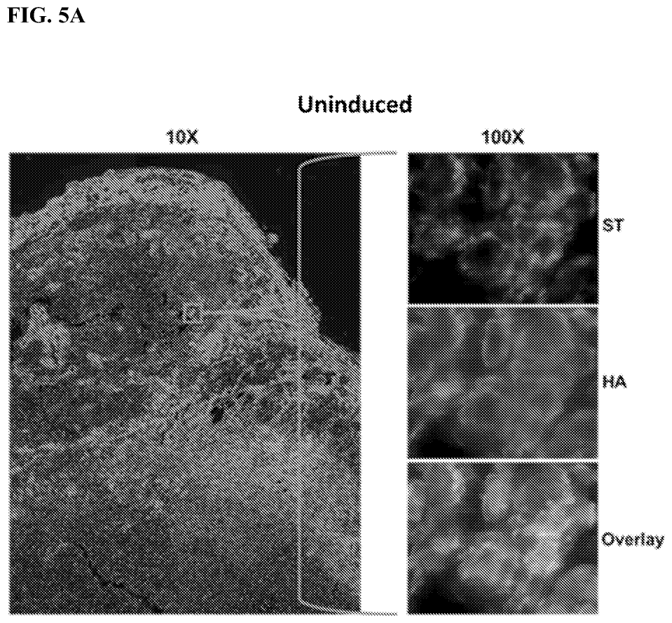

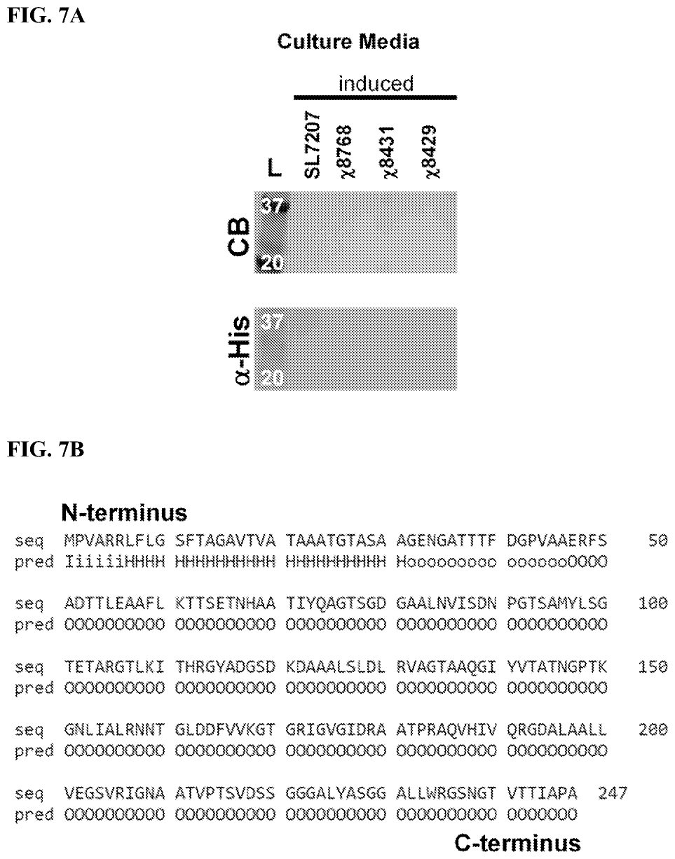

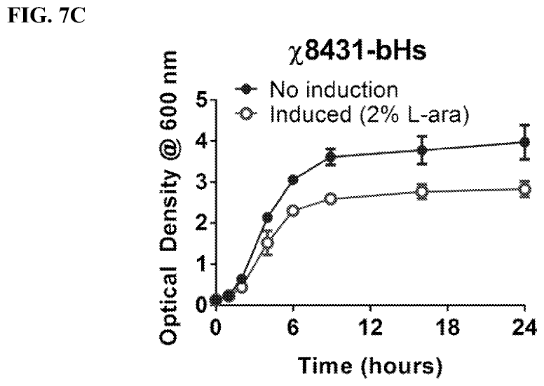

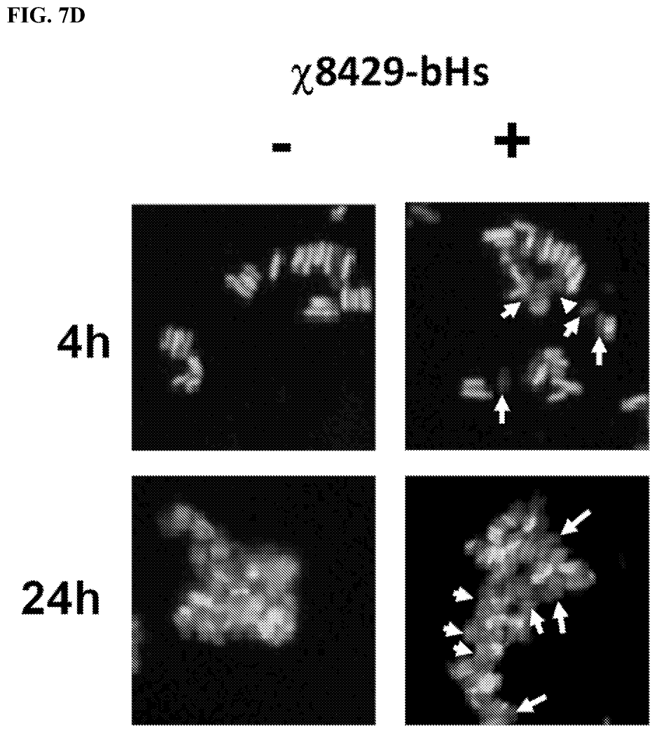

Based on its viability following induction and ability to efficiently degrade purified HA, χ8768-bHs was selected to further determine if bHs-expressing ST could degrade tumor-derived HA. The human pancreatic cancer line PANC-1 was utilized, after confirming it expresses high levels of HA when grown orthotopically in NSG (immune-deficient) mice. First, in vitro HA degradation experiments were performed whereby PANC-1 tumor sections were incubated with pre-induced χ8768-bHs. Overnight incubation of PANC-1 tumor sections with pre-induced χ8768-bHs resulted in dramatic degradation of HA compared to sections incubated with PBS or uninduced χ8768-bHs (FIG. 4). Similar degradation experiments were performed using the PC-3 prostate cancer cell line, which secretes high levels of HA while in culture, and also observed considerable depletion of HA by induced χ8768-bHs (FIG. 7). These results strongly suggest that χ8768 expressing bHs could be used universally to degrade tumor-derived HA in various cancer types.

Systemically-Delivered χ8768-bHs Degrades HA in Orthotopic PDAC Tumors

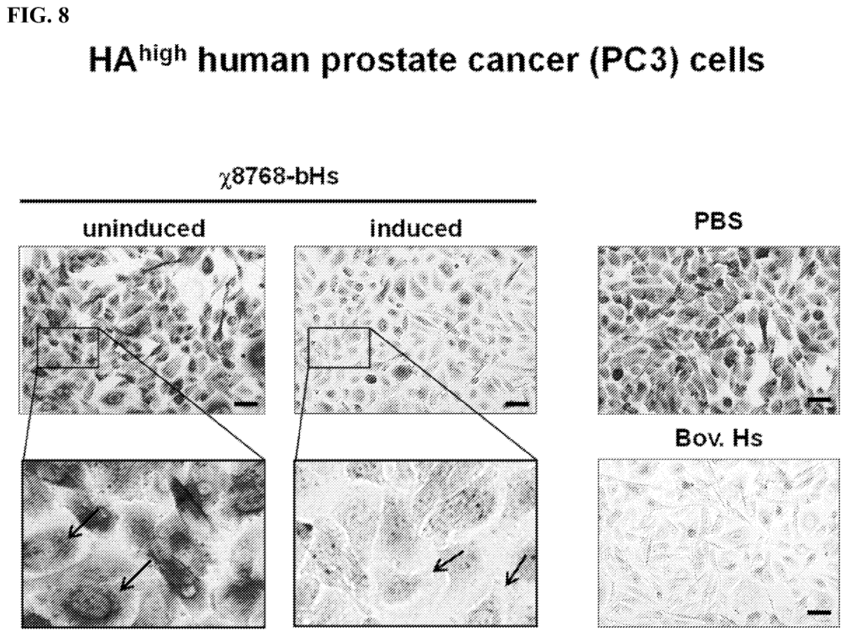

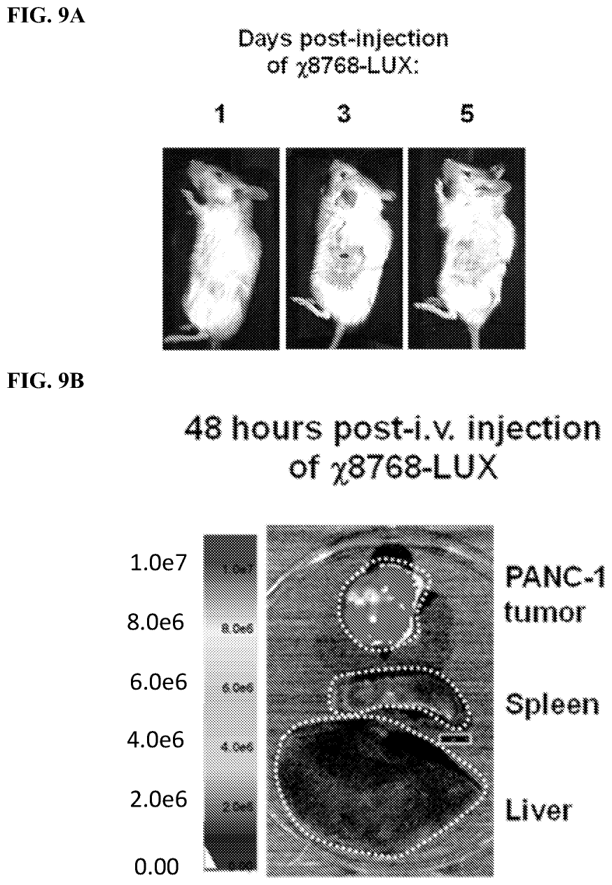

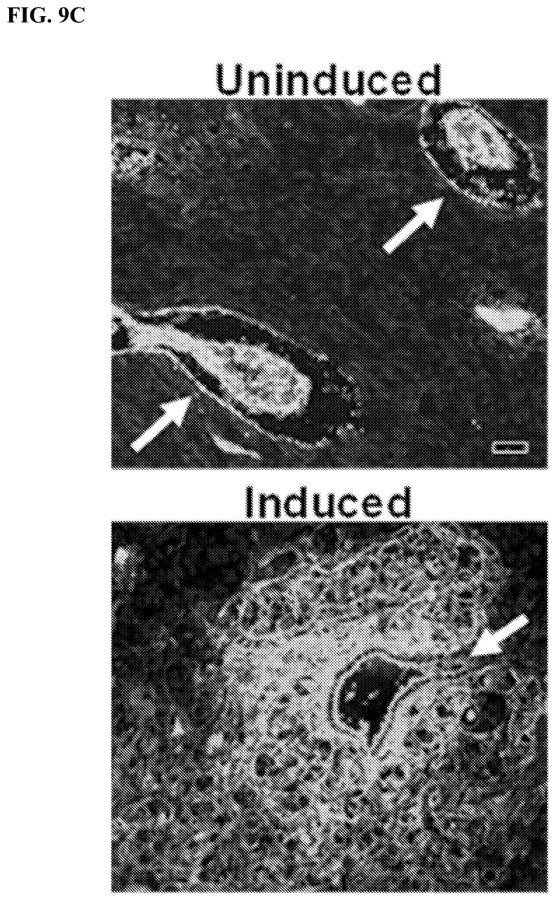

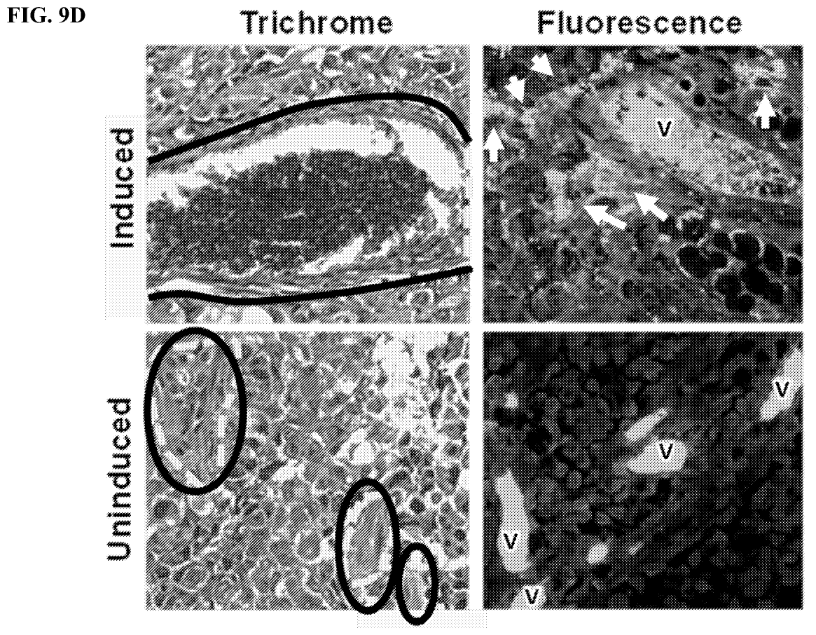

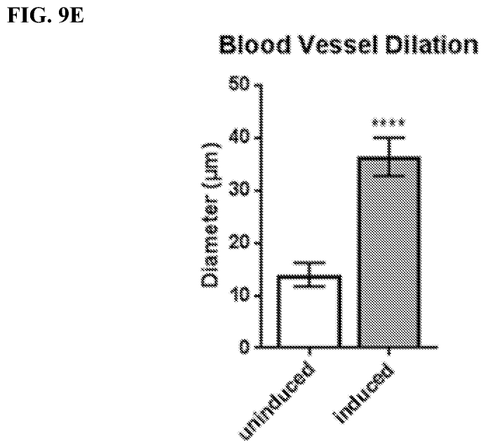

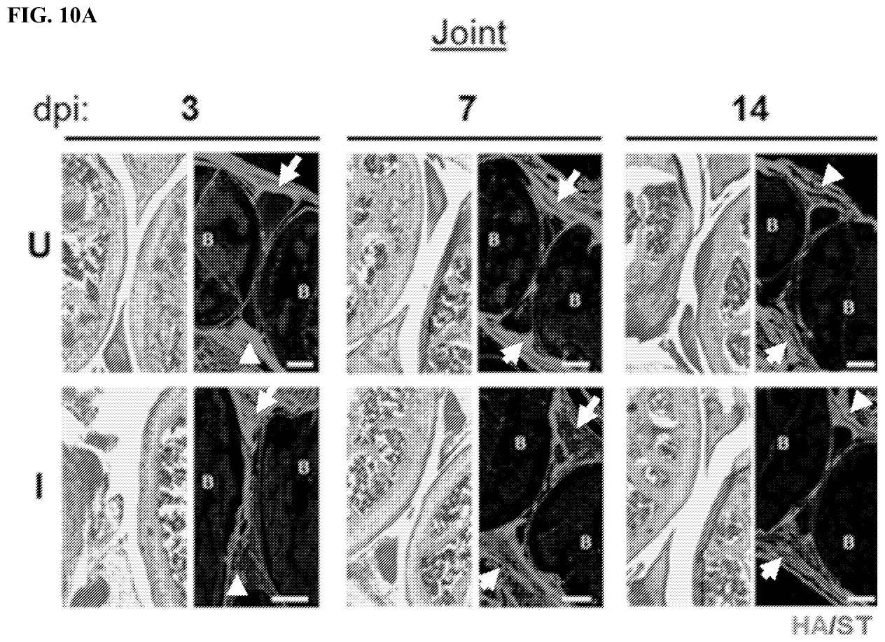

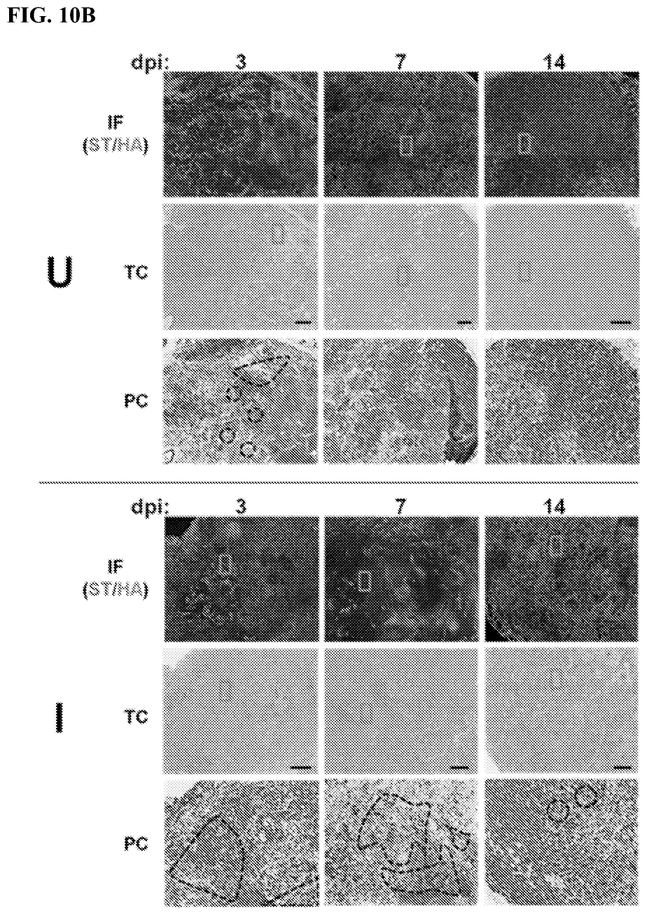

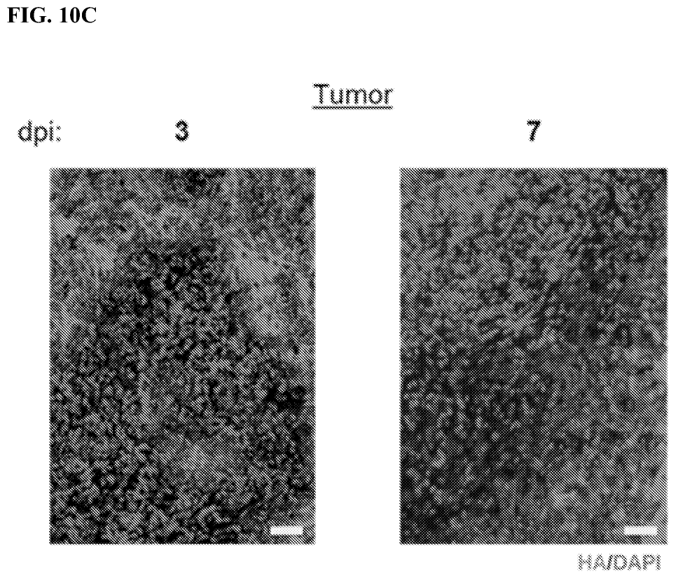

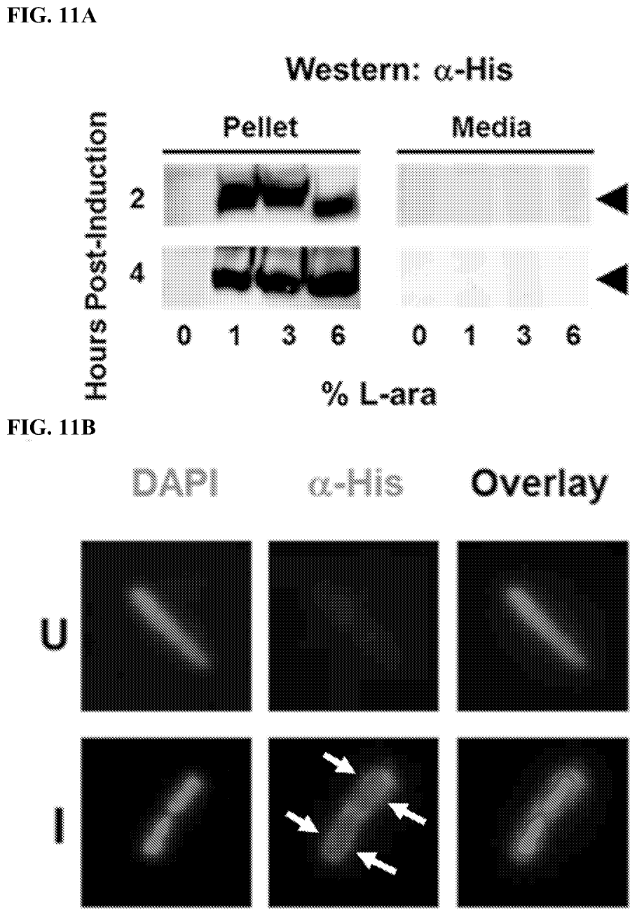

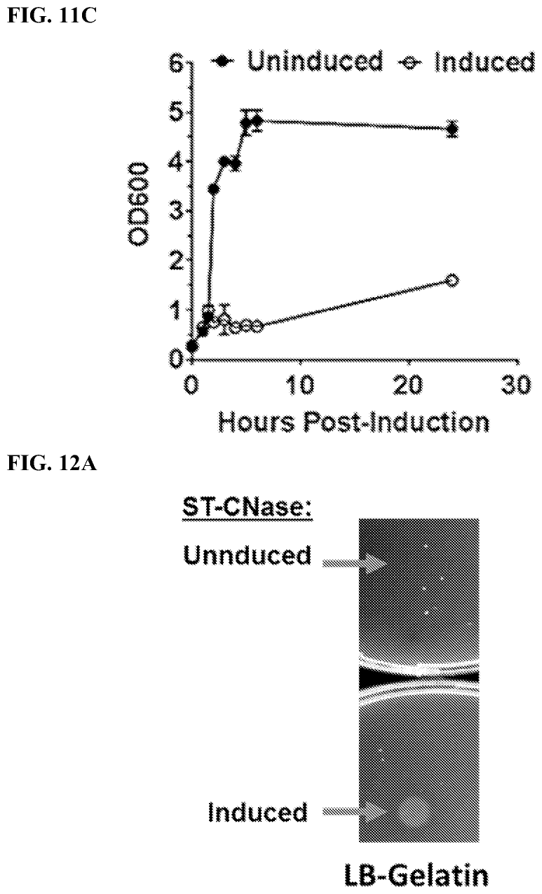

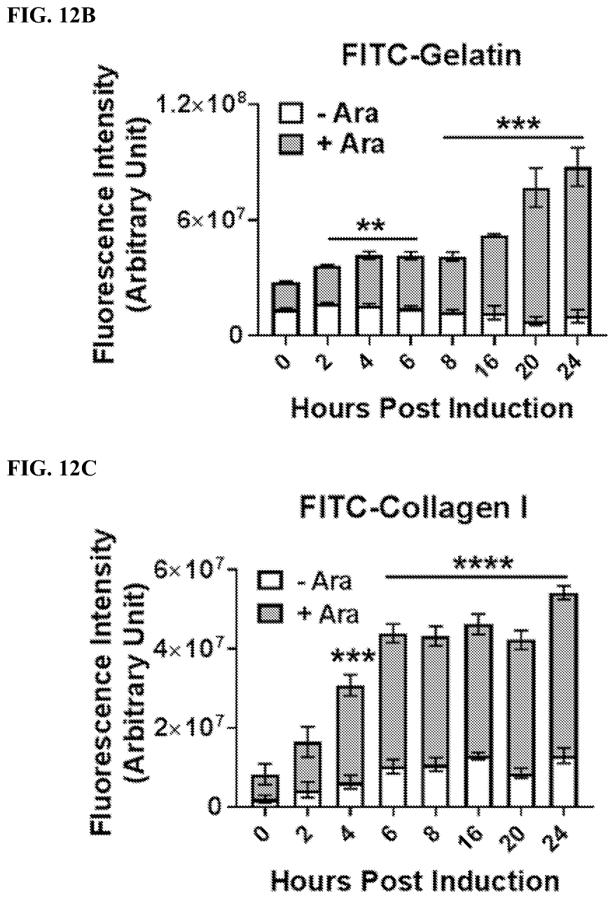

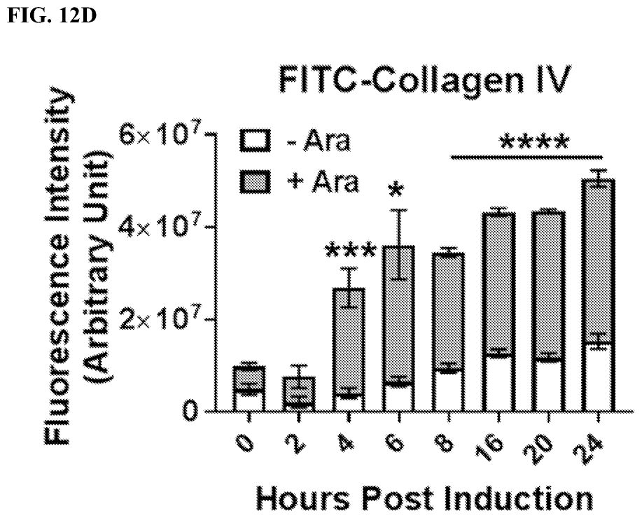

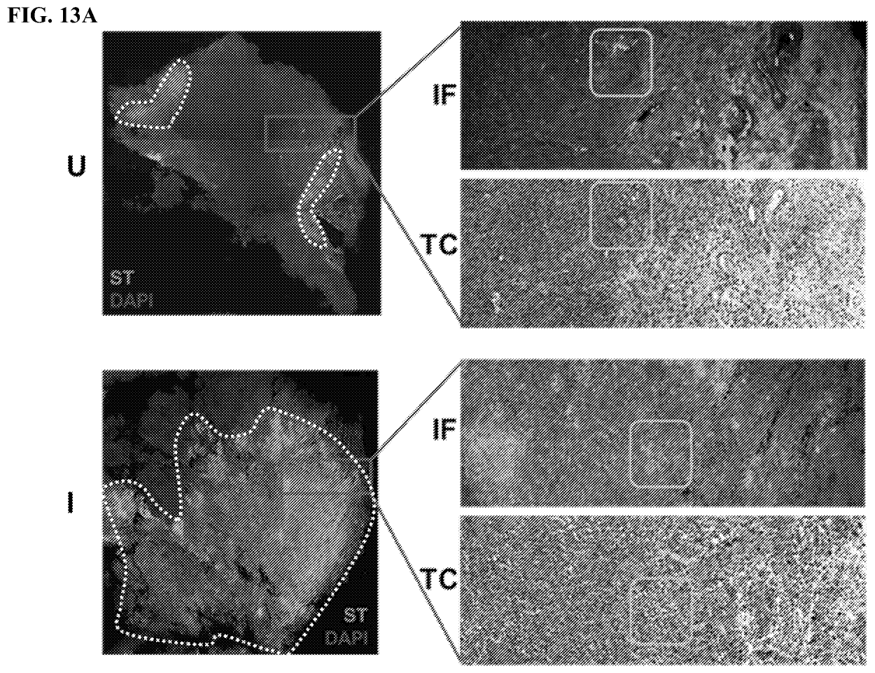

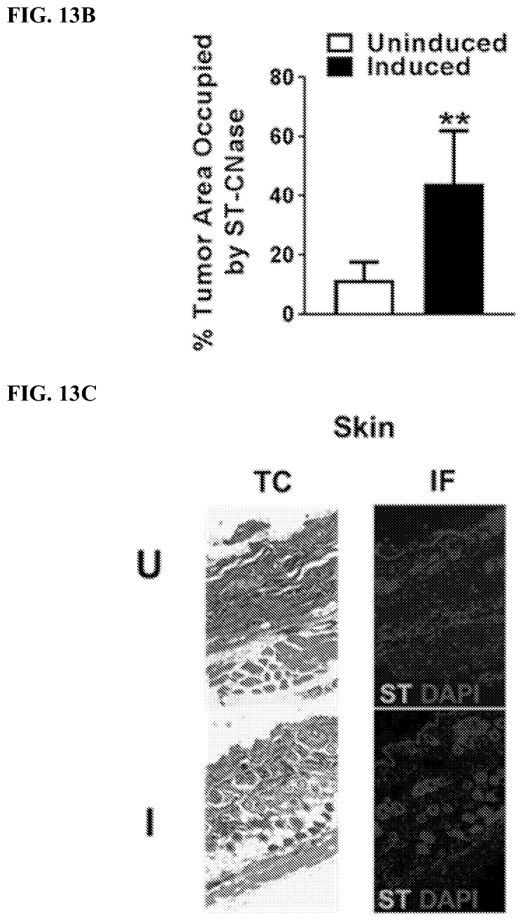

Next, the ability of χ8768-bHs to colonize and deplete HA was determined in orthotopically implanted PANC-1 tumors when delivered intravenously into mice. First, verification that the χ8768 strain was capable of colonizing PANC-1 tumors utilizing a constitutive bacterial reporter gene construct encoding the bioluminescent LUX operon (50) was undertaken. When recombinant χ8768 encoding LUX (χ8768-LUX) was injected intravenously into NSG mice bearing orthotopically implanted PANC-1 tumors (>250 mm₃) observations showed bioluminescence localized to the area of the tumor, which was detected on day 3 and was undetectable on day 5 (FIG. 8A). To further verify tumor-specific colonization by χ8768-LUX, tumor, spleen and liver were isolated following i.v. injection and measured bioluminescence for each tissue type. Indeed, χ8768-LUX was highly concentrated in tumor tissue while completely absent in both spleen and liver (FIG. 8B). These results suggest that χ8768-bHs is capable of colonizing orthotopic PANC-1 tumors after systemic administration and that tumor-specific colonization is achieved by day 2, which would represent an ideal time point for induction of bHs activity.

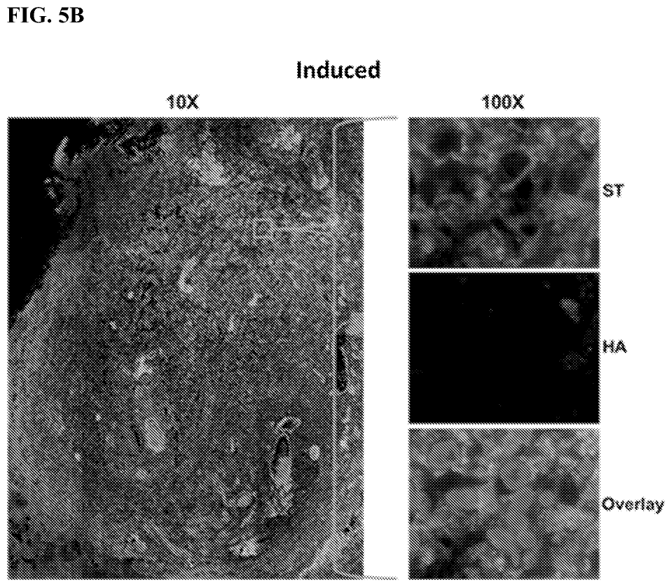

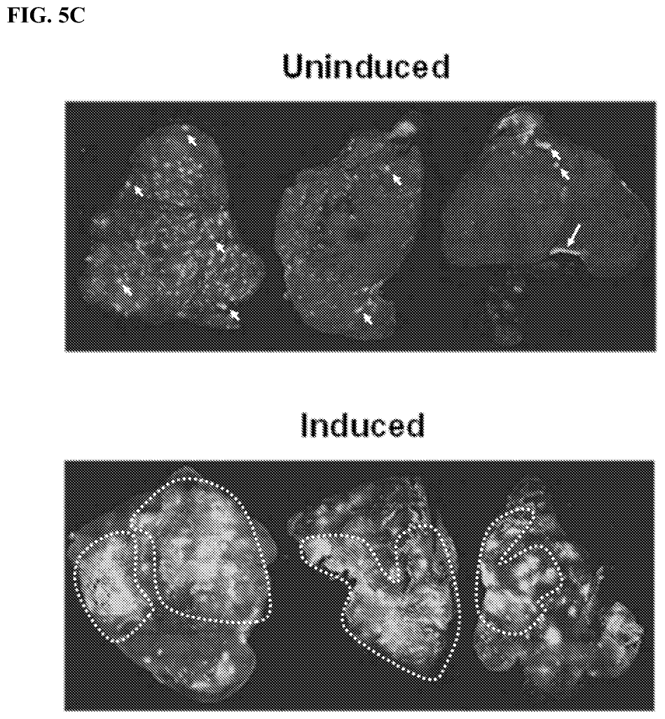

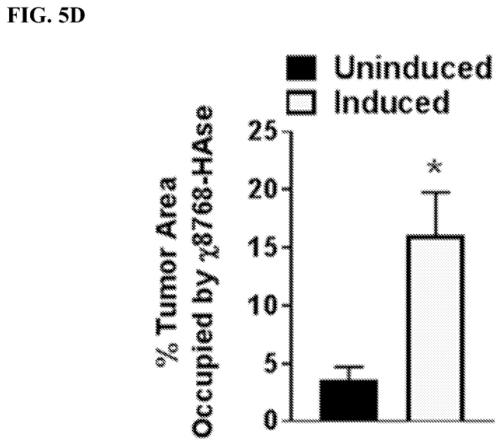

Thus, NSG mice with orthotopic PANC-1 tumors (>250 mm₃) were intravenously administered 2.5×10⁶ CFUs of χ8768-bHs and then induced 2 days later by a single intraperitoneal injection of 240 mg of L-arabinose per mouse. PANC-1 tumors were excised 24 after induction (in the methods and materials you wrote 16 hours) and sectioned and stained for both HA and ST. As shown in FIG. 5A, tumors from mice receiving only χ8768-bHs (uninduced) were characterized by limited (punctate) ST colonization and high HA deposition in areas of ST colonization. In contrast, mice administered χ8768-bHs and L-arabinose (induced) resulted in dramatic degradation of HA, particularly within areas colonized by χ8768-bHs (FIG. 5B). Remarkably, greater diffusion of χ8768-bHs (p<0.05, t test) was also observed under induced conditions (FIG. 5C, D). Diffusion of χ8768-bHs was observed from a number of duct-like structures (likely blood vessels) in tumors of induced mice, which were predominantly devoid of HA. In uninduced mice, these structures were less prevalent but when observed, ST were found within the duct but had not colonized the surrounding tissue which showed high HA staining (FIG. 8C). Through trichrome staining, the blood vessels are more easily visualized by the presence of collagen and red blood cells, revealing a predominance of larger, open vessels in induced mice compared to a majority of closed vessels in uninduced mice, reminiscent of the concentrated punctate staining of ST colonization in these tumors (FIG. 8D, E). Altogether, these data strongly suggest that χ8768-bHs is capable of effectively degrading tumor-derived HA in vivo to decrease interstitial pressure and facilitate delivery of agents as large as ST, in which a single bacterium can measure 5 μm in length and reach a molecular weight in the hundreds of gigadaltons (for example 51-55).

Discussion

Hyaluronidase administration has been shown to enhance the efficacy of gemcitabine and nAb-paclitaxel in PDAC tumor models and has had some clinical benefit in PDAC patients (for example 56, 57). However, the high risk of adverse effects associated with systemically delivered hyaluronidase still presents major concerns due to ECM degradation in healthy tissues. To reduce risk, lower doses of hyaluronidase must be given, which may not necessarily maximize the therapeutic efficacy of chemotherapy. BHs-ST is the first example of a systemically delivered ECM-degrading agent that can focus its enzymatic activity strictly to tumor tissue, potentially maximizing HA degradation and therapeutic drug delivery. Data herein established that bHs is anchored to the surface of ST, reducing the likelihood of systemic off-tumor effects resulting from circulating bHs. Previous studies have also shown that bHs expressed by S. koganeiensis has a unique specificity to HA, further reducing the risk of degrading other major ECM components in healthy tissue. Furthermore, the data showed replication of attenuated ST in tumor tissue, suggesting that initial input of bHs-ST could be amplified in tumors in a few days before induction of bHs to cause maximal HA degradation. The ability of attenuated ST to extensively colonize tumors also increases the window for therapeutic intervention and maximal delivery. The duration of HA degradation, the types and sizes of therapeutic agents being delivered and whether multiple administrations are possible will be ongoing studies to determine the overall utility of bHs-ST.

ST-based cancer therapy has been shown to regress tumors in pre-clinical models and this regression is heavily dependent on the ability of ST to colonize tumors (for example 58, 59). The first attenuated ST to enter clinical trials, VNP20009, was administered to patients with metastatic melanoma and head and neck cancer (for example 30). Virtually no tumor regression was observed in any patients receiving VNP20009 and only a small number of patients had tumors colonized by ST. The disclosure herein shows the ability to express bHs in ST could significantly increase tumor colonization and efficacy of ST-based therapies. Indeed, previous work utilizing a PEGylated hyaluronidase (PEGPH20) significantly improved colonization and anti-tumor efficacy of our ST-based therapeutic.

The number of therapies being developed to treat cancer (nanoparticles, antibody-based therapies, chemotherapeutic combinations, viruses and bacteria) continues to rise and, therefore, strategies to improve penetration of these agents is also required. Currently, only a small number of ECM-targeting agents can claim to improve drug delivery (8, 18). By developing ECM-degrading agents that are more tumor-specific, one can increase the number of potential therapeutic combinations to maximize efficacy while minimizing toxicity. The data herein shows that bHs-ST is capable of targeting tumor-derived HA in PDAC tumors and increasing penetration of large particles. While bHs-ST may only be limited to patients with HA-high tumors, such as those treated with PEGPH20 (56), the ST platform described in this work could be used to develop other ECM-targeting strategies with more universal application and benefit in PDAC patients (for example 28). This suggests that delivery of relatively large particles such as bacteria could be enhanced through the use of ECM-degrading agents. Therefore, bHs-ST pre-treatment could easily be combined with virotherapy or antibody based therapies to improve their delivery.

Sequences

Salmonella typhimurium codon-optimized
DNA sequences:
Streptomyces koganeinsis hyaluronidase
(750 bp):
ATGCCGGTTGCGCGTCGTCTGTTCCTGGGCTCTTTCACCGCGGGCGC
GGTTACCGTTGCGACCGCGGCGGCGACCGGCACCGCGTCTGCGGCGG
GCGAAAACGGCGCGACCACCACCTTCGACGGCCCGGTTGCGGCGGAA
CGTTTCTCTGCGGACACCACCCTGGAAGCGGCGTTCCTGAAAACCAC
CTCTGAAACCAACCACGCGGCGACCATCTACCAGGCGGGCACCTCTG
GCGACGGCGCGGCGCTGAACGTTATCTCTGACAACCCGGGCACCTCT
GCGATGTACCTGTCTGGCACCGAAACCGCGCGTGGCACCCTGAAAAT
CACCCACCGTGGCTACGCGGACGGCTCTGACAAAGACGCGGCGGCGC
TGTCTCTGGACCTGCGTGTTGCGGGCACCGCGGCGCAGGGCATCTAC
GTTACCGCGACCAACGGCCCGACCAAAGGCAACCTGATCGCGCTGCG
TAACAACACCGGCCTGGACGACTTCGTTGTTAAAGGCACCGGCCGTA
TCGGCGTTGGCATCGACCGTGCGGCGACCCCGCGTGCGCAGGTTCAC
ATCGTTCAGCGTGGCGACGCGCTGGCGGCGCTGCTGGTTGAAGGCTC
TGTTCGTATCGGCAACGCGGCGACCGTTCCGACCTCTGTTGACTCTT
CTGGCGGCGGCGCGCTGTACGCGTCTGGCGGCGCGCTGCTGTGGCGT
GGCTCTAACGGCACCGTTACCACCATCGCGCCGGCGTAATAGTGA
Streptomyces omiyaensis trypsin-like
protease (783 bp):
ATGCAGAAAAACCGTCTGGTTCGTACCCTGCAGAAACTGGCGGCGGC
GGGCGCGGTTGCGCTGGCGGCGCTGTCTCTGCAGCCGGTTTCTTCTG
CGACCGCGGCGCCGAACCCGGTTGTTGGCGGCACCCGTGCGGCGCAG
GGCGAATTTCCGTGGATGGTTCGTCTGTCTATGGGCTGCGGCGGCTC
TCTGATCACCCCGCAGGTTGTTCTGACCGCGGCGCACTGCGTTGGCG
CGACCGGCAACAACACCTCTATCACCGCGACCGCGGGCGTTGTTGAC
CTGCAGTCTTCTTCTGCGATCAAAGTTCGTTCTACCAAAATCTACCG
TGCGCCGGGCTACAACGGCAAAGGCAAAGACTGGGCGCTGATCAAAC
TGGCGTCTCCGATCACCTCTCTGCCGACCCTGAAACTGGCGGAAACC
ACCGCGTACAACTCTGGCACCTTCACCGTTGCGGGCTGGGGCGCGGC
GCGTGAAGGCGGCGGCCAGCAGCGTTACCTGCTGAAAGCGAACGTTC
CGTTCGTTTCTGACGCGTCTTGCCAGGCGTCTTACGGCTCTGACCTG
GTTCCGTCTGAAGAAATCTGCGCGGGCTACCCGCAGGGCGGCGTTGA
CACCTGCCAGGGCGACTCTGGCGGCCCGATGTTCCGTAAAGACAACG
CGGGCGCGTGGGTTCAGGTTGGCATCGTTTCTTGGGGCCAGGGCTGC
GCGCGTCCGGACTACCCGGGCGTTTACACCGAAGTTTCTACCTTCGC
GGCGGCGATCAAATCTGCGGCGGCGACCCTG

### REFERENCES

1. Lee J J, Huang J, England C G, McNally L R, Frieboes H B. Predictive modeling of in vivo response to gemcitabine in pancreatic cancer. PLoS computational biology. 2013; 9(9):e1003231. doi: 10.1371/journal.pcbi.1003231. PubMed PMID: 24068909; PubMed Central PMCID: PMC3777914.
2. Han H, Von Hoff D D. SnapShot: pancreatic cancer. Cancer cell. 2013; 23(3):424-e1. doi: 10.1016/j.ccr.2013.03.008. PubMed PMID: 23518352; PubMed Central PMCID: PMC3638898.
3. Sirri E, Castro F A, Kieschke J, Jansen L, Emrich K, Gondos A, et al. Recent Trends in Survival of Patients With Pancreatic Cancer in Germany and the United States. Pancreas. 2016; 45(6):908-14. doi: 10.1097/MPA.0000000000000588. PubMed PMID: 26745860.
4. Uzunparmak B, Sahin I H. Pancreatic cancer microenvironment: a current dilemma. Clinical and translational medicine. 2019; 8(1):2. doi: 10.1186/s40169-019-0221-1. PubMed PMID: 30645701.
5. Liang C, Shi S, Meng Q, Liang D, Ji S, Zhang B, et al. Complex roles of the stroma in the intrinsic resistance to gemcitabine in pancreatic cancer: where we are and where we are going. Experimental & molecular medicine. 2017; 49(12):e406. doi: 10.1038/emm.2017.255. PubMed PMID: 29611542; PubMed Central PMCID: PMC5750480.
6. Bahrami A, Khazaei M, Bagherieh F, Ghayour-Mobarhan M, Maftouh M, Hassanian S M, et al. Targeting stroma in pancreatic cancer: Promises and failures of targeted therapies. Journal of cellular physiology. 2017; 232(11):2931-7. doi: 10.1002/jcp.25798. PubMed PMID: 28083912.
7. Erkan M, Hausmann S, Michalski C W, Fingerle A A, Dobritz M, Kleeff J, et al. The role of stroma in pancreatic cancer: diagnostic and therapeutic implications. Nature reviews Gastroenterology & hepatology. 2012; 9(8):454-67. doi: 10.1038/nrgastro.2012.115. PubMed PMID: 22710569.
8. Jacobetz M A, Chan D S, Neesse A, Bapiro T E, Cook N, Frese K K, et al. Hyaluronan impairs vascular function and drug delivery in a mouse model of pancreatic cancer. Gut. 2013; 62(1):112-20. doi: 10.1136/gutjnl-2012-302529. PubMed PMID: 22466618; PubMed Central PMCID: PMC3551211.
9. Li X, Ma Q, Xu Q, Duan W, Lei J, Wu E. Targeting the cancer-stroma interaction: a potential approach for pancreatic cancer treatment. Current pharmaceutical design. 2012; 18(17):2404-15. PubMed PMID: 22372501; PubMed Central PMCID: PMC3414722.
10. Auvinen P, Rilla K, Tumelius R, Tammi M, Sironen R, Soini Y, et al. Hyaluronan synthases (HAS1-3) in stromal and malignant cells correlate with breast cancer grade and predict patient survival. Breast cancer research and treatment. 2014; 143(2):277-86. doi: 10.1007/s10549-013-2804-7. PubMed PMID: 24337597.
11. Udabage L, Brownlee G R, Waltham M, Blick T, Walker E C, Heldin P, et al. Antisense-mediated suppression of hyaluronan synthase 2 inhibits the tumorigenesis and progression of breast cancer. Cancer research. 2005; 65(14):6139-50. doi: 10.1158/0008-5472.CAN-04-1622. PubMed PMID: 16024615.
12. Josefsson A, Adamo H, Hammarsten P, Granfors T, Stattin P, Egevad L, et al. Prostate cancer increases hyaluronan in surrounding nonmalignant stroma, and this response is associated with tumor growth and an unfavorable outcome. The American journal of pathology. 2011; 179(4):1961-8. doi: 10.1016/j.ajpath.2011.06.005. PubMed PMID: 21854754; PubMed Central PMCID: PMC3181394.
13. Huang H, Brekken R A. The Next Wave of Stroma-Targeting Therapy in Pancreatic Cancer. Cancer research. 2019; 79(2):328-30. doi: 10.1158/0008-5472.CAN-18-3751. PubMed PMID: 30647067.
14. Cun X, Ruan S, Chen J, Zhang L, Li J, He Q, et al. A dual strategy to improve the penetration and treatment of breast cancer by combining shrinking nanoparticles with collagen depletion by losartan. Acta biomaterialia. 2016; 31:186-96. doi: 10.1016/j.actbio.2015.12.002. PubMed PMID: 26675124.
15. Schuster L, Seifert O, Vollmer S, Kontermann R E, Schlosshauer B, Hartmann H. Immunoliposomes for Targeted Delivery of an Antifibrotic Drug. Molecular pharmaceutics. 2015; 12(9):3146-57. doi: 10.1021/acs.molpharmaceut.5b00012. PubMed PMID: 26181293.
16. Khawar I A, Kim J H, Kuh H J. Improving drug delivery to solid tumors: priming the tumor microenvironment. Journal of controlled release: official journal of the Controlled Release Society. 2015; 201:78-89. doi: 10.1016/j.jconrel.2014.12.018. PubMed PMID: 25526702.
17. Fukumura D, Jain R K. Tumor microvasculature and microenvironment: targets for anti-angiogenesis and normalization. Microvascular research. 2007; 74(2-3):72-84. doi: 10.1016/j.mvr.2007.05.003. PubMed PMID: 17560615; PubMed Central PMCID: PMC2100036.
18. Elahi-Gedwillo K Y, Carlson M, Zettervall J, Provenzano P P. Antifibrotic Therapy Disrupts Stromal Barriers and Modulates the Immune Landscape in Pancreatic Ductal Adenocarcinoma. Cancer research. 2019; 79(2):372-86. doi: 10.1158/0008-5472.CAN-18-1334. PubMed PMID: 30401713; PubMed Central PMCID: PMC6335156.
19. Kim J H, Shin B C, Park W S, Lee J, Kuh H J. Antifibrotic effects of pentoxifylline improve the efficacy of gemcitabine in human pancreatic tumor xenografts. Cancer science. 2017; 108(12):2470-7. doi: 10.1111/cas.13405. PubMed PMID: 28940685; PubMed Central PMCID: PMC5715266.
20. Buckway B, Wang Y, Ray A, Ghandehari H. Overcoming the stromal barrier for targeted delivery of HPMA copolymers to pancreatic tumors. International journal of pharmaceutics. 2013; 456(1):202-11. doi: 10.1016/j.ijpharm.2013.07.067. PubMed PMID: 23933441.
21. Baumgartner G, Gomar-Hoss C, Sakr L, Ulsperger E, Wogritsch C. The impact of extracellular matrix on the chemoresistance of solid tumors—experimental and clinical results of hyaluronidase as additive to cytostatic chemotherapy. Cancer letters. 1998; 131(1):85-99. PubMed PMID: 9839623.
22. Beckenlehner K, Bannke S, Spruss T, Bernhardt G, Schonenberg H, Schiess W.

Hyaluronidase enhances the activity of adriamycin in breast cancer models in vitro and in vivo. Journal of cancer research and clinical oncology. 1992; 118(8):591-6. PubMed PMID: 1517281.
23. Hoffman P, Meyer K, Linker A. Transglycosylation during the mixed digestion of hyaluronic acid and chondroitin sulfate by testicular hyaluronidase. The Journal of biological chemistry. 1956; 219(2):653-63. PubMed PMID: 13319286.
24. Stern R, Jedrzejas M J. Hyaluronidases: their genomics, structures, and mechanisms of action. Chemical reviews. 2006; 106(3):818-39. doi: 10.1021/cr050247k. PubMed PMID: 16522010; PubMed Central PMCID: PMC2547145.
25. Pavan M, Beninatto R, Galesso D, Panfilo S, Vaccaro S, Messina L, et al. A new potential spreading factor: Streptomyces koganeiensis hyaluronidase. A comparative study with bovine testes hyaluronidase and recombinant human hyaluronidase of the HA degradation in ECM. Biochimica et biophysica acta. 2016; 1860(4):661-8. doi: 10.1016/j.bbagen.2015.12.024. PubMed PMID: 26723173.
26. Hynes W L, Walton S L. Hyaluronidases of Gram-positive bacteria. FEMS microbiology letters. 2000; 183(2):201-7. doi: 10.1111/j.1574-6968.2000.tb08958.x. PubMed PMID: 10675584.
27. Kawaguchi K, Miyake K, Zhao M, Kiyuna T, Igarashi K, Miyake M, et al. Tumor targeting Salmonella typhimurium A1-R in combination with gemcitabine (GEM) regresses partially GEM-resistant pancreatic cancer patient-derived orthotopic xenograft (PDOX) nude mouse models. Cell cycle. 2018; 17(16):2019-26. doi: 10.1080/15384101.2018.1480223. PubMed PMID: 29963961; PubMed Central PMCID: PMC6224272.
28. Manuel E R, Chen J, D'Apuzzo M, Lampa M G, Kaltcheva T I, Thompson C B, et al. Salmonella-Based Therapy Targeting Indoleamine 2,3-Dioxygenase Coupled with Enzymatic Depletion of Tumor Hyaluronan Induces Complete Regression of Aggressive Pancreatic Tumors. Cancer Immunol Res. 2015; 3(9):1096-107. doi: 10.1158/2326-6066.CIR-14-0214. PubMed PMID: 26134178; PubMed Central PMCID: PMC4561205.
29. Jia L J, Xu H M, Ma D Y, Hu Q G, Huang X F, Jiang W H, et al. Enhanced therapeutic effect by combination of tumor-targeting Salmonella and endostatin in murine melanoma model. Cancer Biol Ther. 2005; 4(8):840-5. PubMed PMID: 16210914.
30. Toso J F, Gill V J, Hwu P, Marincola F M, Restifo N P, Schwartzentruber D J, et al. Phase I study of the intravenous administration of attenuated Salmonella typhimurium to patients with metastatic melanoma. J Clin Oncol. 2002; 20(1):142-52. doi: 10.1200/jco.2002.20.1.142. PubMed PMID: 11773163; PubMed Central PMCID: PMC2064865.
31. Pawelek J M, Low K B, Bermudes D. Tumor-targeted Salmonella as a novel anticancer vector. Cancer Res. 1997; 57(20):4537-44. PubMed PMID: 9377566.
32. Orlando Z, Lengers I, Melzig M F, Buschauer A, Hensel A, Jose J. Autodisplay of Human Hyaluronidase Hyal-1 on Escherichia coli and Identification of Plant-Derived Enzyme Inhibitors. Molecules. 2015; 20(9):15449-68. doi: 10.3390/molecules200915449. PubMed PMID: 26343612.
33. Kaessler A, Olgen S, Jose J. Autodisplay of catalytically active human hyaluronidase hPH-20 and testing of enzyme inhibitors. European journal of pharmaceutical sciences: official journal of the European Federation for Pharmaceutical Sciences. 2011; 42(1-2):138-47. doi: 10.1016/j.ejps.2010.11.004. PubMed PMID: 21075205.
34. Hofinger E S, Spickenreither M, Oschmann J, Bernhardt G, Rudolph R, Buschauer A. Recombinant human hyaluronidase Hyal-1: insect cells versus Escherichia coli as expression system and identification of low molecular weight inhibitors. Glycobiology. 2007; 17(4):444-53. doi: 10.1093/glycob/cwm003. PubMed PMID: 17227790.
35. Bollen W S, Gunn B M, Mo H, Lay M K, Curtiss R, 3rd. Presence of wild-type and attenuated Salmonella enterica strains in brain tissues following inoculation of mice by different routes. Infection and immunity. 2008; 76(7):3268-72. doi: 10.1128/IAI.00244-08. PubMed PMID: 18474649; PubMed Central PMCID: PMC2446729.
36. Xiong G, Husseiny M I, Song L, Erdreich-Epstein A, Shackleford G M, Seeger R C, et al. Novel cancer vaccine based on genes of Salmonella pathogenicity island 2. Int J Cancer. 2010; 126(11):2622-34. doi: 10.1002/ijc.24957. PubMed PMID: 19824039; PubMed Central PMCID: PMC2993175.
37. Hoiseth S K, Stocker B A. Aromatic-dependent Salmonella typhimurium are non-virulent and effective as live vaccines. Nature. 1981; 291(5812):238-9. PubMed PMID: 7015147.
38. Evans D T, Chen L M, Gillis J, Lin K C, Harty B, Mazzara G P, et al. Mucosal priming of simian immunodeficiency virus-specific cytotoxic T-lymphocyte responses in rhesus macaques by the Salmonella type III secretion antigen delivery system. Journal of virology. 2003; 77(4):2400-9. PubMed PMID: 12551977; PubMed Central PMCID: PMC141091.
39. Johnson S A, Ormsby M J, Wall D M. Draft Genome Sequence of the Tumor-Targeting Salmonella enterica Serovar Typhimurium Strain SL7207. Genome announcements. 2017; 5(5):e01591-16. doi: 10.1128/genomeA.01591-16. PubMed PMID: 28153911.
40. Hart M E, Tsang L H, Deck J, Daily S T, Jones R C, Liu H, et al. Hyaluronidase expression and biofilm involvement in Staphylococcus aureus UAMS-1 and its sarA, agr and sarA agr regulatory mutants. Microbiology. 2013; 159(Pt 4):782-91. doi: 10.1099/mic.0.065367-0. PubMed PMID: 23393148; PubMed Central PMCID: PMC3709827.
41. Oueslati N, Leblanc P, Harscoat-Schiavo C, Rondags E, Meunier S, Kapel R, et al. CTAB turbidimetric method for assaying hyaluronic acid in complex environments and under cross-linked form. Carbohydrate polymers. 2014; 112:102-8. doi: 10.1016/j.carbpol.2014.05.039. PubMed PMID: 25129722.
42. Tseng W W, Winer D, Kenkel J A, Choi O, Shain A H, Pollack J R, et al. Development of an orthotopic model of invasive pancreatic cancer in an immunocompetent murine host. Clinical cancer research: an official journal of the American Association for Cancer Research. 2010; 16(14):3684-95. doi: 10.1158/1078-0432.CCR-09-2384. PubMed PMID: 20534740; PubMed Central PMCID: PMC3085509.
43. Guzman L M, Belin D, Carson M J, Beckwith J. Tight regulation, modulation, and high-level expression by vectors containing the arabinose PBAD promoter. Journal of bacteriology. 1995; 177(14):4121-30. PubMed PMID: 7608087; PubMed Central PMCID: PMC177145.
44. Matusiak M, Van Opdenbosch N, Vande Walle L, Sirard J C, Kanneganti T D, Lamkanfi M. Flagellin-induced NLRC4 phosphorylation primes the inflammasome for activation by NAIPS. Proceedings of the National Academy of Sciences of the United States of America. 2015; 112(5):1541-6. doi: 10.1073/pnas.1417945112. PubMed PMID: 25605939; PubMed Central PMCID: PMC4321312.
45. Chou K C, Shen H B. Cell-PLoc: a package of Web servers for predicting subcellular localization of proteins in various organisms. Nature protocols. 2008; 3(2):153-62. doi: 10.1038/nprot.2007.494. PubMed PMID: 18274516.
46. Yu N Y, Laird M R, Spencer C, Brinkman F S. PSORTdb—an expanded, auto-updated, user-friendly protein subcellular localization database for Bacteria and Archaea. Nucleic acids research. 2011; 39(Database issue):D241-4. doi: 10.1093/nar/gkq1093. PubMed PMID: 21071402; PubMed Central PMCID: PMC3013690.
47. Tusnady G E, Simon I. The HMMTOP transmembrane topology prediction server.

Bioinformatics. 2001; 17(9):849-50. PubMed PMID: 11590105.
48. Kasibhatla S, Amarante-Mendes G P, Finucane D, Brunner T, Bossy-Wetzel E, Green D R. Acridine Orange/Ethidium Bromide (AO/EB) Staining to Detect Apoptosis. CSH protocols. 2006; 2006(3). doi: 10.1101/pdb.prot4493. PubMed PMID: 22485874.
49. Zhao Y, Xing Q, Janjanam J, He K, Long F, Low K B, et al. Facile electrochemical synthesis of antimicrobial TiO(2) nanotube arrays. International journal of nanomedicine. 2014; 9:5177-87. doi: 10.2147/IJN.S65386. PubMed PMID: 25429214; PubMed Central PMCID: PMC4243507.
50. Karsi A, Lawrence M L. Broad host range fluorescence and bioluminescence expression vectors for Gram-negative bacteria. Plasmid. 2007; 57(3):286-95. doi: 10.1016/j.plasmid.2006.11.002. PubMed PMID: 17207855.
51. Weniger M, Honselmann K C, Liss A S. The Extracellular Matrix and Pancreatic Cancer: A Complex Relationship. Cancers. 2018; 10(9). doi: 10.3390/cancers10090316. PubMed PMID: 30200666; PubMed Central PMCID: PMC6162452.
52. DuFort C C, DelGiorno K E, Carlson M A, Osgood R J, Zhao C, Huang Z, et al. Interstitial Pressure in Pancreatic Ductal Adenocarcinoma Is Dominated by a Gel-Fluid Phase. Biophysical journal. 2016; 110(9):2106-19. doi: 10.1016/j.bpj.2016.03.040. PubMed PMID: 27166818; PubMed Central PMCID: PMC4939548.
53. Chauhan V P, Boucher Y, Ferrone C R, Roberge S, Martin J D, Stylianopoulos T, et al. Compression of pancreatic tumor blood vessels by hyaluronan is caused by solid stress and not interstitial fluid pressure. Cancer cell. 2014; 26(1):14-5. doi: 10.1016/j.ccr.2014.06.003. PubMed PMID: 25026209; PubMed Central PMCID: PMC4381566.
54. Provenzano P P, Hingorani S R. Hyaluronan, fluid pressure, and stromal resistance in pancreas cancer. British journal of cancer. 2013; 108(1):1-8. doi: 10.1038/bjc.2012.569. PubMed PMID: 23299539; PubMed Central PMCID: PMC3553539.
55. Fabrega A, Vila J. Salmonella enterica serovar Typhimurium skills to succeed in the host: virulence and regulation. Clinical microbiology reviews. 2013; 26(2):308-41. doi: 10.1128/CMR.00066-12. PubMed PMID: 23554419; PubMed Central PMCID: PMC3623383.
56. Doherty G J, Tempero M, Corrie P G. HALO-109-301: a Phase III trial of PEGPH20 (with gemcitabine and nab-paclitaxel) in hyaluronic acid-high stage IV pancreatic cancer. Future oncology. 2018; 14(1):13-22. doi: 10.2217/fon-2017-0338. PubMed PMID: 29235360.
57. Hingorani S R, Zheng L, Bullock A J, Seery T E, Harris W P, Sigal D S, et al. HALO 202: Randomized Phase II Study of PEGPH20 Plus Nab-Paclitaxel/Gemcitabine Versus Nab-Paclitaxel/Gemcitabine in Patients With Untreated, Metastatic Pancreatic Ductal Adenocarcinoma. Journal of clinical oncology: official journal of the American Society of Clinical Oncology. 2018; 36(4):359-66. doi: 10.1200/JCO.2017.74.9564. PubMed PMID: 29232172.
58. Felgner S, Kocijancic D, Frahm M, Heise U, Rohde M, Zimmermann K, et al. Engineered Salmonella enterica serovar Typhimurium overcomes limitations of anti-bacterial immunity in bacteria-mediated tumor therapy. Oncoimmunology. 2018; 7(2):e1382791. doi: 10.1080/2162402X.2017.1382791. PubMed PMID: 29308303; PubMed Central PMCID: PMC5749626.
59. Yu B, Shi L, Zhang B Z, Zhang K E, Peng X, Niu H B, et al. Obligate anaerobic Salmonella typhimurium strain YB1 treatment on xenograft tumor in immunocompetent mouse model. Oncol Lett. 2015; 10(2):1069-74. doi: 10.3892/01.2015.3302. PubMed PMID: 26622627; PubMed Central PMCID: PMC4509430.
60. Rodriguez et al. “Live attenuated salmonella vaccines against Mycobacterium tuberculosis with antigen delivery via the type iii secretion system”, infection and imminity, 2012.
61. Arrach, N., Zhao, M., Porwollik, S., Hoffman, R. M. & McClelland, M. Salmonella promoters preferentially activated inside tumors. Cancer Res. 2008; 68(12):4827-4832. Epub 2008/06/19. doi: 10.1158/0008-5472.CAN-08-0552. PMID: 18559530.
62. Ryan, R. M. et al. Bacterial delivery of a novel cytolysin to hypoxic areas of solid tumors. Gene therapy. 2009; 16(3):329-339. Epub 2009/01/30. doi: 10.1038/gt.2008.188. PMID: 19177133.
63. Mengesha, A. et al. Development of a flexible and potent hypoxia-inducible promoter for tumor-targeted gene expression in attenuated Salmonella. Cancer Biol Ther. 2006; 5(9):1120-1128. Epub 2006/07/21. doi: 10.4161/cbt.5.9.2951. PMID: 16855381.
64. Muller, P., Chikkaballi, D. & Hensel, M. Functional dissection of SseF, a membrane-integral effector protein of intracellular Salmonella enterica. PloS one. 2012; 7(4):e35004. Epub 2012/04/25. doi: 10.1371/journal.pone.0035004. PMID: 22529968; PMCID: PMC3329539.
65. Xiong G, Husseiny M I, Song L, Erdreich-Epstein A, Shackleford G M, Seeger R C, et al. Novel cancer vaccine based on genes of Salmonella pathogenicity island 2. Int J Cancer 2010; 126:2622-34.
66. Pawelek J M, Low K B, Bermudes D. Tumor-targeted Salmonella as a novel anticancer vector. Cancer Res 1997; 57:4537-44.
67. Low, K. B. et al. Construction of VNP20009: a novel, genetically stable antibiotic-sensitive strain of tumor-targeting Salmonella for parenteral administration in humans. Methods Mol Med. 2004; 90:47-60. PMID: 14657558.
68. Lee S H, Mizutani N, Mizutani M, Luo Y, Zhou H, Kaplan C, et al. Endoglin (CD105) is a target for an oral DNA vaccine against breast cancer. Cancer Immunol Immunother 2006; 55:1565-74.
69. Hohmann E. L., Oletta C. A., Miller S. I. (1996) Evaluation of a phoP/phoQ-deleted, aroA-deleted live oral Salmonella typhi vaccine strain in human volunteers. Vaccine 14:19024.
70. Hoiseth S K, Stocker B A. Aromatic-dependent Salmonella typhimurium are non-virulent and effective as live vaccines. Nature 1981; 291:238-9
71. Johnson S A, Ormsby M J, Wall D M. Draft Genome Sequence of the Tumor-Targeting Salmonella enterica Serovar Typhimurium Strain SL7207. Genome announcements 2017; 5:e01591-16.
72. Bollen W S, Gunn B M, Mo H, Lay M K, Curtiss R, 3rd. Presence of wild-type and attenuated Salmonella enterica strains in brain tissues following inoculation of mice by different routes. Infection and immunity 2008; 76:3268-72.
73. Evans D T, Chen L M, Gillis J, Lin K C, Harty B, Mazzara G P, et al. Mucosal priming of simian immunodeficiency virus-specific cytotoxic T-lymphocyte responses in rhesus macaques by the Salmonella type III secretion antigen delivery system. Journal of virology 2003; 77:2400-9.
74. Zheng, Jin Hai, and Jung-Joon Min. “Targeted Cancer Therapy Using Engineered Salmonella typhimurium.” Chonnam medical journal vol. 52,3 (2016): 173-84. doi:10.4068/cmj.2016.52.3.173
75. Pavan M, Beninatto R, Galesso D, Panfilo S, Vaccaro S, Messina L, et al. A new potential spreading factor: Streptomyces koganeiensis hyaluronidase. A comparative study with bovine testes hyaluronidase and recombinant human hyaluronidase of the HA degradation in ECM. Biochimica et biophysica acta 2016; 1860:661-8
76. Uesugi Y, Arima J, Usuki H, Iwabuchi M, Hatanaka T. Two bacterial collagenolytic serine proteases have different topological specificities. Biochim Biophys Acta. 2008; 1784(4):716-726. doi:10.1016/j.bbapap.2008.01.017
77. Zhang, Y. Z., Ran, L. Y., Li, C. Y., & Chen, X. L. (2015). Diversity, Structures, and Collagen-Degrading Mechanisms of Bacterial Collagenolytic Proteases. Applied and environmental microbiology, 81(18), 6098-6107. https://doi.org/10.1128/AEM.00883-15
78. Sequence: UniProtKB—A0A0U2E2J7 (A0A0U2E2J7_9ACTN)—Streptomyces koganeiensis Hyaluronidase
79. Sequence: UniProtKB—C9K160 (C9K160_9ACTN)—Streptomyces omiyaensis Trypsin-like protease

### P-Embodiments

Embodiment P-1. An attenuated facultative anaerobic bacterium comprising a nucleic acid molecule encoding a recombinant extracellular matrix degrading enzyme operably linked to a promoter.

Embodiment P-2. The attenuated facultative anaerobic bacterium of Embodiment P-1 selected from Salmonella bongori, Salmonella choleraesuis, Salmonella enterica, Salmonella enteritidis, Salmonella paratyphi, Salmonella typhi, Salmonella typhimurium, Vibrio cholerae, Vibrio fischeri, Escherichia coli, Shigella boydii, Shigella dysenteriae, Shigella flexneri, Shigella sonnei, Lactobacillus bulgaricus, Listeria monocytogenes, Enterococcus faecalis, Enterococcus gallolyticus, Enterococcus faecium, and Streptococcus pyogenes.

Embodiment P-3. The attenuated facultative anaerobic bacterium of Embodiment P-2, wherein the Salmonella typhimurium is selected from MVP728, YS1646 (VNP20009), RE88, LH430, SL7207, χ8429, χ8431 and χ8768.

Embodiment P-4. The attenuated facultative anaerobic bacterium of one of Embodiments P-1 to P-3 wherein the recombinant extracellular matrix degrading enzyme is selected from a human extracellular matrix degrading enzyme, a bacterial extracellular matrix degrading enzyme, and a parasitic extracellular matrix degrading enzyme.

Embodiment P-5. The attenuated facultative anaerobic bacterium of one of Embodiments P-1 to P-4, wherein the recombinant extracellular matrix degrading enzyme is selected from matrix metalloproteinase, collagenase, hyaluronidase, chondroitinase, heparatinase, cathepsin, lyase, trypsin, protease, plasmin, and urokinase.

Embodiment P-6. The attenuated facultative anaerobic bacterium of Embodiment P-4, wherein the recombinant extracellular matrix degrading enzyme is hyaluronidase.

Embodiment P-7. The attenuated facultative anaerobic bacterium of Embodiment P-6, wherein the hyaluronidase is bacterial hyaluronidase.

Embodiment P-8. The attenuated facultative anaerobic bacterium of Embodiment P-7, wherein the bacterial hyaluronidase is from Streptomyces koganeiensis, Streptomyces hyaluronlyticus, Staphylococcus aureus, Streptococcus pyogenes and Clostridium perfringens.

Embodiment P-9. The attenuated facultative anaerobic bacterium of one of Embodiments P-1 to P-5, wherein the recombinant extracellular matrix degrading enzyme is collagenase.

Embodiment P-10. The attenuated facultative anaerobic bacterium of one of Embodiments P-1 to P-9, wherein the promoter is an inducible promoter.

Embodiment P-11. The attenuated facultative anaerobic bacterium of Embodiment P-10, wherein the promoter is selected from a pLac promoter, pTac promoter, a tetracycline-controlled promoter, and a pBAD promoter.

Embodiment P-12. The attenuated facultative anaerobic bacterium of any of Embodiments P-1 to P-9, wherein the promoter is a tumor-specific promoter.

Embodiment P-13. The attenuated facultative anaerobic bacterium of Embodiment P-2, wherein the promoter is a hypoxia-inducible bacterial promoter.

Embodiment P-14. The attenuated facultative anaerobic bacterium of Embodiment P-3, wherein the hypoxia-inducible bacterial promoter is FF+20*, HIP1, or selected from those regulating expression of spflE, hcp, menD, ansB, mltD, glpA, glpT, and pepT.

Embodiment P-15. A method of treating a tumor in a subject comprising administering to the subject an effective amount of an attenuated facultative anaerobic bacterium comprising a nucleic acid molecule encoding a recombinant extracellular matrix degrading enzyme operably linked to a promoter.

Embodiment P-16. The method of Embodiment P-15, wherein the tumor is a solid tumor.

Embodiment P-17. The method of any of Embodiments P-15 to P-16, wherein the tumor is selected from pancreatic, breast, prostate, skin, lung, and abdomen tumor.

Embodiment P-18. The method of any of Embodiment P-15 to P-17, wherein the tumor is a pancreatic ductal adenocarcinoma.

Embodiment P-19. The method of any of Embodiments P-15 to P-18, wherein the attenuated facultative anaerobic bacterium is selected from Salmonella bongori, Salmonella choleraesuis, Salmonella enterica, Salmonella enteritidis, Salmonella paratyphi, Salmonella typhi, Salmonella typhimurium, Vibrio cholerae, Vibrio fischeri, Escherichia coli, Shigella boydii, Shigella dysenteriae, Shigella flexneri, Shigella sonnei. Lactobacillus bulgaricus, Listeria monocytogenes, Enterococcus faecalis, Enterococcus gallolyticus, Enterococcus faecium, and Streptococcus pyogenes.

Embodiment P-20. The method of Embodiment P-19, wherein the Salmonella typhimurium is selected from MVP728, YS1646 (VNP20009), RE88, LH430, SL7207, χ8429, χ8431 and χ8768.

Embodiment P-21. The method of any of Embodiments P-15 to P-20, wherein the recombinant extracellular matrix degrading enzyme is selected from a human extracellular matrix degrading enzyme, a bacterial extracellular matrix degrading enzyme, and a parasitic extracellular matrix degrading enzyme.

Embodiment P-22. The method of any of Embodiments P-15 to P-21, wherein the recombinant extracellular matrix degrading enzyme is selected from matrix metalloproteinase, collagenase, hyaluronidase, chondroitinase, heparatinase, cathepsin, lyase, trypsin, protease, plasmin, and urokinase.

Embodiment P-23. The method of Embodiment P-22, wherein the recombinant extracellular matrix degrading enzyme is hyaluronidase.

Embodiment P-24. The method of Embodiment P-23, wherein the hyaluronidase is bacterial hyaluronidase.

Embodiment P-25. The method of Embodiment P-24, wherein the bacterial hyaluronidase is from Streptomyces koganeiensis, Streptomyces hyaluronlyticus, Staphylococcus aureus, Streptococcus pyogenes and Clostridium perfringens.

Embodiment P-26. The method of any one of Embodiments P-15 to P-22, wherein the recombinant extracellular matrix degrading enzyme is collagenase.

Embodiment P-27. The method of any one of Embodiments P-15 to P-26, wherein the promoter is an inducible promoter.

Embodiment P-28. The method of Embodiment P-27, wherein the promoter is selected from a pLac promoter, a pTac promoter, a tetracycline-controlled promoter, and a pBAD promoter.

Embodiment P-29. The method of any one of Embodiment P-15 to P-26, wherein the promoter is a tumor-specific promoter.

Embodiment P-30. The method of Embodiment P-29, wherein the promoter is a hypoxia-inducible bacterial promoter.

Embodiment P-31. The method of Embodiment P-30, wherein the hypoxia-inducible bacterial promoter is selected from FF+20*, HIP1, or those regulating expression of spflE, hcp, menD, ansB, mltD, glpA, glpT, and pepT.

Embodiment P-32. A method of treating tumor in a subject, comprising the step of administering to the subject a combined effective amount of an attenuated facultative anaerobic bacteria and a chemotherapeutic agent, wherein the bacteria comprises a nucleic acid molecule encoding a recombinant extracellular matrix degrading enzyme operably linked to a promoter.

Embodiment P-33. The method of Embodiment P-32, wherein the tumor is a solid tumor.

Embodiment P-34. The method of any of Embodiments P-32 to P-33, wherein the tumor is selected from pancreatic, breast, and prostate tumor.

Embodiment P-35. The method of any of claims 32-34, wherein the tumor is a pancreatic ductal adenocarcinoma.

Embodiment P-36. The method of any of Embodiment P-32 to P-35, wherein the bacteria is a species selected from Salmonella bongori, Salmonella choleraesuis, Salmonella enterica, Salmonella enteritidis, Salmonella paratyphi, Salmonella typhi, Salmonella typhimurium, Vibrio cholerae, Vibrio fischeri, Escherichia coli, Shigella boydii, Shigella dysenteriae, Shigella flexneri, Shigella sonnei. Lactobacillus bulgaricus, Listeria monocytogenes, Enterococcus faecalis, Enterococcus gallolyticus, Enterococcus faecium, and Streptococcus pyogenes.

Embodiment P-37. The method of Embodiment P-36, wherein the Salmonella typhimurium is a strain selected from strains MVP728, YS1646 (VNP20009), RE88, LH430, SL7207, χ8429, χ8431 and χ8768.

Embodiment P-38. The method of any of Embodiments P-32 to P-37, wherein the recombinant extracellular matrix degrading enzyme is selected from a human extracellular matrix degrading enzyme, a bacterial extracellular matrix degrading enzyme, and a parasitic extracellular matrix degrading enzyme.

Embodiment P-39. The method of any of Embodiments P-32 to P-38, wherein the recombinant extracellular matrix degrading enzyme is selected from matrix metalloproteinase, collagenase, hyaluronidase, chondroitinase, heparatinase, cathepsin, lyase, trypsin, protease, plasmin, and urokinase.

Embodiment P-40. The method of Embodiment P-39, wherein the recombinant extracellular matrix degrading enzyme is hyaluronidase.

Embodiment P-41. The method of Embodiment P-40, wherein the hyaluronidase is bacterial hyaluronidase.

Embodiment P-42. The method of Embodiment P-41, wherein the bacterial hyaluronidase is from Streptomyces koganeiensis, Streptomyces hyaluronlyticus, Staphylococcus aureus, Streptococcus pyogenes and Clostridium perfringens.

Embodiment P-43. The method of any one of Embodiments P-32 to P-39, wherein the recombinant extracellular matrix degrading enzyme is collagenase.

Embodiment P-44. The method of any one of Embodiment P-32 to P-43, wherein the promoter is an inducible promoter.

Embodiment P-45. The method of Embodiment P-44, wherein the promoter selected is a pLac promoter, a pTac promoter, a tetracycline-controlled promoter, and a pBAD promoter.

Embodiment P-46. The method of any one of Embodiments P-32 to P-43, wherein the promoter is a tumor-specific promoter.

Embodiment P-47. The method of Embodiment P-46, wherein the promoter is a hypoxia-inducible bacterial promoter.

Embodiment P-48. The method of Embodiment P-47, wherein the hypoxia-inducible bacterial promoter is selected from FF+20*, HIP1, or those regulating expression of spflE, hcp, menD, ansB, mltD, glpA, glpT, and pepT.

Embodiment P-49. The method of any one of Embodiment P-32 to P-48, wherein the chemotherapeutic agent is selected from Abraxane, asparaginase, bleomycin, busulfan carmustine, chlorambucil, cladribine, CPT-11, cyclophosphamide, cytarabine, dacarbazine, daunorubicin, dexamethasone, doxorubicin (commonly referred to as Adriamycin), etoposide, fludarabine, folfirinox, 5-fluorouracil, gemcitabine, hydroxyurea, idarubicin, ifosfamide, interferon-α (native or recombinant), levamisole, and lomustine, mechlorethamine, melphalan, mercaptopurine, methotrexate, mitomycin, mitoxantrone, paclitaxel, pentostatin, prednisone, procarbazine, tamoxife, taxol-related compounds, 6-thiogaunine, topotecan, vinblastine, and vincristine.

What is claimed is:

1. An attenuated facultative anaerobic bacterium comprising a nucleic acid molecule encoding a recombinant extracellular matrix degrading enzyme operably linked to a promoter.

2. The attenuated facultative anaerobic bacterium of claim 1 selected from Salmonella bongori, Salmonella choleraesuis, Salmonella enterica, Salmonella enteritidis, Salmonella paratyphi, Salmonella typhi, Salmonella typhimurium, Vibrio cholerae, Vibrio fischeri, Escherichia coli, Shigella boydii, Shigella dysenteriae, Shigella flexneri, Shigella sonnei, Lactobacillus bulgaricus, Listeria monocytogenes, Enterococcus faecalis, Enterococcus gallolyticus, Enterococcus faecium, and Streptococcus pyogenes.

3. The attenuated facultative anaerobic bacterium of claim 2, wherein the Salmonella typhimurium is selected from MVP728, YS1646 (VNP20009), RE88, LH430, SL7207, χ8429, χ8431 and χ8768.

4. The attenuated facultative anaerobic bacterium of one of claims 1 to 3 wherein the recombinant extracellular matrix degrading enzyme is selected from a human extracellular matrix degrading enzyme, a bacterial extracellular matrix degrading enzyme, and a parasitic extracellular matrix degrading enzyme.

5. The attenuated facultative anaerobic bacterium of one of claims 1 to 4, wherein the recombinant extracellular matrix degrading enzyme is selected from matrix metalloproteinase, collagenase, hyaluronidase, chondroitinase, heparatinase, cathepsin, lyase, trypsin, protease, plasmin, and urokinase.

6. The attenuated facultative anaerobic bacterium of claim 5, wherein recombinant extracellular matrix degrading enzyme is a protease encoded by a codon optimized nucleic acid comprising SEQ ID NO.: 2.

7. The attenuated facultative anaerobic bacterium of claim 5, wherein the recombinant extracellular matrix degrading enzyme is hyaluronidase.

8. The attenuated facultative anaerobic bacterium of claim 7, wherein the hyaluronidase is bacterial hyaluronidase.

9. The attenuated facultative anaerobic bacterium of claim 8, wherein the bacterial hyaluronidase is from Streptomyces koganeiensis, Streptomyces hyaluronlyticus, Staphylococcus aureus, Streptococcus pyogenes and Clostridium perfringens.

10. The attenuated facultative anaerobic bacterium of claim 9, wherein the bacterial hyaluronidase is encoded by a codon optimized nucleic acid sequence comprising SEQ ID NO.: 1.

11. The attenuated facultative anaerobic bacterium of one of claims 1-5, wherein the recombinant extracellular matrix degrading enzyme is collagenase.

12. The attenuated facultative anaerobic bacterium of one of claims 1-11, wherein the promoter is an inducible promoter.

13. The attenuated facultative anaerobic bacterium of claim 12, wherein the promoter is selected from a pLac promoter, pTac promoter, a tetracycline-controlled promoter, and a pBAD promoter.

14. The attenuated facultative anaerobic bacterium of any of claims 1-11, wherein the promoter is a tumor-specific promoter.

15. The attenuated facultative anaerobic bacterium of claim 14, wherein the promoter is a hypoxia-inducible bacterial promoter.

16. The attenuated facultative anaerobic bacterium of claim 15, wherein the hypoxia-inducible bacterial promoter is FF+20*, HIP1, or selected from those regulating expression of spflE, hcp, menD, ansB, mltD, glpA, glpT, and pepT.

17. A method of treating a tumor in a subject comprising administering to the subject an effective amount of an attenuated facultative anaerobic bacterium comprising a nucleic acid molecule encoding a recombinant extracellular matrix degrading enzyme operably linked to a promoter.

18. The method of claim 17, wherein the tumor is a solid tumor.

19. The method of any of claims 17-18, wherein the tumor is selected from pancreatic, breast, prostate, skin, lung, and abdomen tumor.

20. The method of any of claims 17-19, wherein the tumor is a pancreatic ductal adenocarcinoma.

21. The method of any of claims 17-20, wherein the attenuated facultative anaerobic bacterium is selected from Salmonella bongori, Salmonella choleraesuis, Salmonella enterica, Salmonella enteritidis, Salmonella paratyphi, Salmonella typhi, Salmonella typhimurium, Vibrio cholerae, Vibrio fischeri, Escherichia coli, Shigella boydii, Shigella dysenteriae, Shigella flexneri, Shigella sonnei. Lactobacillus bulgaricus, Listeria monocytogenes, Enterococcus faecalis, Enterococcus gallolyticus, Enterococcus faecium, and Streptococcus pyogenes.

22. The method of claim 21, wherein the Salmonella typhimurium is selected from MVP728, YS1646 (VNP20009), RE88, LH430, SL7207, χ8429, χ8431 and χ8768.

23. The method of any of claims 17-22, wherein the recombinant extracellular matrix degrading enzyme is selected from a human extracellular matrix degrading enzyme, a bacterial extracellular matrix degrading enzyme, and a parasitic extracellular matrix degrading enzyme.

24. The method of any of claims 17-23, wherein the recombinant extracellular matrix degrading enzyme is selected from matrix metalloproteinase, collagenase, hyaluronidase, chondroitinase, heparatinase, cathepsin, lyase, trypsin, protease, plasmin, and urokinase.

25. The method of claim 24, wherein the recombinant extracellular matrix degrading enzyme is a protease encoded by a codon optimized nucleic acid comprising SEQ ID NO.: 2.

26. The method of claim 24, wherein the recombinant extracellular matrix degrading enzyme is hyaluronidase.

27. The method of claim 26, wherein the hyaluronidase is bacterial hyaluronidase.

28. The method of claim 27, wherein the bacterial hyaluronidase is from Streptomyces koganeiensis, Streptomyces hyaluronlyticus, Staphylococcus aureus, Streptococcus pyogenes and Clostridium perfringens.

29. The method of claim 28, wherein the bacterial hyaluronidase is encoded by a codon optimized nucleic acid sequence comprising SEQ ID NO.: 1.

30. The method of any one of claims 17-24, wherein the recombinant extracellular matrix degrading enzyme is collagenase.

31. The method of any one of claims 17-30, wherein the promoter is an inducible promoter.

32. The method of claim 31, wherein the promoter is selected from a pLac promoter, a pTac promoter, a tetracycline-controlled promoter, and a pBAD promoter.

33. The method of any one of claims 17-30, wherein the promoter is a tumor-specific promoter.

34. The method of claim 33, wherein the promoter is a hypoxia-inducible bacterial promoter.

35. The method of claim 34, wherein the hypoxia-inducible bacterial promoter is selected from FF+20*, HIP1, or those regulating expression of spflE, hcp, menD, ansB, mltD, glpA, glpT, and pepT.

36. A method of treating tumor in a subject, comprising the step of administering to the subject a combined effective amount of an attenuated facultative anaerobic bacteria and a chemotherapeutic agent, wherein the bacteria comprises a nucleic acid molecule encoding a recombinant extracellular matrix degrading enzyme operably linked to a promoter.

37. The method of claim 36, wherein the tumor is a solid tumor.

38. The method of any of claims 36-37, wherein the tumor is selected from pancreatic, breast, and prostate tumor.

39. The method of any of claims 36-38, wherein the tumor is a pancreatic ductal adenocarcinoma.

40. The method of any of claims 36-39, wherein the bacteria is a species selected from Salmonella bongori, Salmonella choleraesuis, Salmonella enterica, Salmonella enteritidis, Salmonella paratyphi, Salmonella typhi, Salmonella typhimurium, Vibrio cholerae, Vibrio fischeri, Escherichia coli, Shigella boydii, Shigella dysenteriae, Shigella flexneri, Shigella sonnei. Lactobacillus bulgaricus, Listeria monocytogenes, Enterococcus faecalis, Enterococcus gallolyticus, Enterococcus faecium, and Streptococcus pyogenes.

41. The method of claim 40, wherein the Salmonella typhimurium is a strain selected from strains MVP728, YS1646 (VNP20009), RE88, LH430, SL7207, χ8429, χ8431 and χ8′768.

42. The method of any of claims 36-41, wherein the recombinant extracellular matrix degrading enzyme is selected from a human extracellular matrix degrading enzyme, a bacterial extracellular matrix degrading enzyme, and a parasitic extracellular matrix degrading enzyme.

43. The method of any of claims 36-42, wherein the recombinant extracellular matrix degrading enzyme is selected from matrix metalloproteinase, collagenase, hyaluronidase, chondroitinase, heparatinase, cathepsin, lyase, trypsin, protease, plasmin, and urokinase.

44. The method of claim 43, wherein the recombinant extracellular matrix degrading enzyme is hyaluronidase.

45. The method of claim 44, wherein the hyaluronidase is bacterial hyaluronidase.

46. The method of claim 45, wherein the bacterial hyaluronidase is from Streptomyces koganeiensis, Streptomyces hyaluronlyticus, Staphylococcus aureus, Streptococcus pyogenes and Clostridium perfringens.

47. The method of claim 46, wherein the bacterial hyaluronidase is encoded by a codon optimized nucleic acid sequence comprising SEQ ID NO.: 1.

48. The method of any one of claims 36-43, wherein the recombinant extracellular matrix degrading enzyme is collagenase.

49. The method of any one of claims 36-48, wherein the promoter is an inducible promoter.

50. The method of claim 49, wherein the promoter selected is a pLac promoter, a pTac promoter, a tetracycline-controlled promoter, and a pBAD promoter.

51. The method of any one of claims 36-47, wherein the promoter is a tumor-specific promoter.

52. The method of claim 51, wherein the promoter is a hypoxia-inducible bacterial promoter.

53. The method of claim 52, wherein the hypoxia-inducible bacterial promoter is selected from FF+20*, HIP1, or those regulating expression of spflE, hcp, menD, ansB, mltD, glpA, glpT, and pepT.

54. The method of any one of claims 36-53, wherein the chemotherapeutic agent is selected from Abraxane, asparaginase, bleomycin, busulfan carmustine, chlorambucil, cladribine, CPT-11, cyclophosphamide, cytarabine, dacarbazine, daunorubicin, dexamethasone, doxorubicin (commonly referred to as Adriamycin), etoposide, fludarabine, folfirinox, 5-fluorouracil, gemcitabine, hydroxyurea, idarubicin, ifosfamide, interferon-α (native or recombinant), levamisole, and lomustine, mechlorethamine, melphalan, mercaptopurine, methotrexate, mitomycin, mitoxantrone, paclitaxel, pentostatin, prednisone, procarbazine, tamoxife, taxol-related compounds, 6-thiogaunine, topotecan, vinblastine, and vincristine.
